(19) United States
(12) Patent Application Publication
(10) Pub. No.: US 2014/0128747 A1
(43) Pub. Date: May 8, 2014

(54) ASSESSING ENDOTHELIAL FUNCTION USING A BLOOD PRESSURE CUFF

(71) Applicant: Jonathan S. Maltz, Oakland, CA (US)

(72) Inventor: Jonathan S. Maltz, Oakland, CA (US)

(73) Assignee: THE REGENTS OF THE UNIVERSITY OF CALIFORNIA, Oakland, CA (US)

(21) Appl. No.: 14/008,299

(22) PCT Filed: Apr. 26, 2012
(86) PCT No.: PCT/US12/35285

§ 371 Date: Dec. 9, 2013

Related U.S. Application Data
(60) Provisional application No. 61/479,304, filed on Apr. 26, 2011.

(51) Int. Cl. A61B 5/02; A61B 17/135; A61B 5/00

(52) U.S. Cl. CPC A61B 5/02007; A61B 5/7246; A61B 17/135; A61B 5/6824; A61B 5/6828
USPC 600/481

(57) ABSTRACT

Methods and apparatus are provided for assessing endothelial function in a mammal. The methods involve applying to the artery a substantially constant external pressure, where the pressure is provided via a cuff adjacent to and/or around a region of the mammal's body; determining, over the course of one or more cardiac cycles, changes in pressure in the cuff resulting from cardiac activity of the mammal to establish a baseline value for a parameter related to endothelial function in the mammal; applying a stimulus to the mammal; determining, over the course of one or more cardiac cycles, changes in pressure in the cuff resulting from cardiac activity of the mammal to establish a stimulus-effected value for a parameter related to endothelial function in the mammal; wherein differences in the baseline value and the stimulus-effected value provide a measure of endothelial function in the mammal.

### CROSS-REFERENCE TO RELATED APPLICATIONS

This application claims benefit of and priority to U.S. Ser. No. 61/479,304, filed on Apr. 26, 2011, which is incorporated herein by reference in its entirety for all purposes.

### STATEMENT OF GOVERNMENTAL SUPPORT

This invention was made with government support under Contract No. DE-AC02-05CH11231 awarded by the U.S. Department of Energy. The government has certain rights in the invention.

### BACKGROUND

Impairment of arterial endothelial function is an early event in atherosclerosis and correlates with all of the major risk factors for cardiovascular disease (CVD). The most widely employed noninvasive measure of endothelial function involves brachial artery (BA) diameter measurement using ultrasound imaging before and after several minutes of blood flow occlusion (Celermajer et al. (1992) The Lancet, 340: 1111-1115). The change in arterial diameter is a measure of flow-mediated vasodilation (FMD). This peripheral measurement correlates strongly with coronary artery endothelial function, a fact which strongly supports its clinical value. However, the high between-laboratory variability of results and cost of instrumentation render this technique unsuitable for routine clinical use.

Endothelial function is both acutely and chronically affected by lifestyle factors that influence CVD risk (Brunner et al. (2005) J. Hypertens., 23: 233-246). Consequently, measures of endothelial function are useful in monitoring response to medication, dietary changes and exercise regimens. Unfortunately, very little work has focused on determining the clinical value of endothelial function measurements for individual patients or on developing measurement methods suitable for routine or continuous monitoring of endothelial function. There are compelling reasons to believe that knowledge of acute variation in endothelial function in an individual is important. Since NO released by the endothelium is a potent inhibitor of leukocyte and monocyte adhesion to the endothelial cell surface, and since adhesion of these cells is widely believed to be a necessary initiating event in atherogenesis (Deanfield et al. (2005) J. Hypertens., 23: 7-17), it is reasonable to infer that the proportion of time that the endothelium is dysfunctional constitutes an important indicator of disease risk. This is the rationale for the development of techniques that are simple and cheap enough to enable regular or continuous measurement of endothelial function.

The two FDA-approved commercially available systems for measuring endothelial function perform measurements that are based on the flow and pulse pressure in resistance vessels (rather than in conduit arteries). The Endo-PAT2000 system from Itamar Medical analyzes the pulse amplitude in the finger before and after application on endothelial stimulus. While about 46% of the observed changes in pulse amplitude are blocked by NO synthase inhibitors, mechanisms other than those mediated by NO significantly contribute towards the response (Nohria et al. (2006) J Appl Physiol, 101(2): 545-548). This is most probably a consequence of the different mechanisms involved in arterial and arteriolar/microvascular vasodilation. Also, the measurement is made in vessels that experience ischemia and the many non-NO-mediated vasodilatory processes that occur under ischemic conditions. It is clinically preferable to perform measurements on arteries such as the brachial artery, the endothelial response of which is highly correlated with that of the coronary arteries (r=0.78, p<0.001, Takase et al. (1998) Am. J. Cardiol., 82(12): 1535-1539). In addition, a review of close to 2,500 studies found that brachial and coronary artery EF have similar power to predict serious cardiovascular events over a follow-up period of 1-92 months (Lerman and Zeiher (2005) Circulation, 111(3): 363-368). The authors of the review assert that “the similar power of coronary and peripheral endothelial dysfunction to predict cardiovascular events and the observation that the cardiovascular events may occur remotely from the site in which the endothelial dysfunction was detected underscore the systemic nature of endothelial dysfunction and its pivotal role in prediction of cardiovascular events.” It is not currently possible to make such strong statements regarding the significance of microvascular endothelial function.

While the largest study (N=1957) of the Endo-PAT system showed encouraging correlations with cardiovascular risk factors and good predictive value of future cardiovascular events, some of the results suggest the influence of potentially serious confounding factors. For example, while it is well known that endothelial function tends to decrease with age, older subjects exhibited better endothelial response according to Endo-PAT (Hamburg et al. (2008) Circulation, 117(19): 2467-2474).

A second approved device is the Vendys system developed by Endothelix, Inc. of Houston Tex. This system measures the cutaneous reactive hyperemic response using hand skin temperature measurement during two minutes of brachial artery occlusion and ensuing RH. During occlusion, skin temperature drops in the distal hand. As blood flow is restored, the temperature increases. Studies indicate that the recovery of skin temperature is slowed in subjects having higher Framingham risk scores and other metrics of CVD and CVD risk. Interestingly, substantial temperature changes are also observed in the contralateral hand that experiences no reactive hyperemic episode. This suggests significant neural involvement in the response. For this reason and the results of Wong et al. (2003) J. Appl. Physiol., 95: 504-510 it is reasonable to predict that this response cannot be blocked by NOS inhibitors.

There is no doubt that these systems provide clinical value and can identify patients with pooled cardiovascular risk factors. However, it is not clear that these systems can do this better than paper-based scoring methods such as the Framingham risk in general populations. It is also highly probable that sympathetic nervous activation is a significant confounding factor in endothelial function measurements based on arteriolar and microvascular responses.

Much stronger evidence exists that peripheral artery endothelial function provides more than simply a correlate of CVD risk factors. Few clinicians would disagree with the statement that evaluation of EF in conduit arteries has more proven clinical value.

A number of patents have issued directed to method and apparatus for evaluating endothelial function.

Whitt et al. (U.S. Pat. No. 6,309,359) appears to describe a method that involves placing an occlusive cuff around a limb (e.g. an arm) of a patient. A fluid, such as air, is pumped into the cuff, and the pressure in the cuff is measured. The pressure variation in the cuff with respect to time is caused by the pump and expansion/contraction of the arm caused by blood being pumped therethrough by the patient's heart. This variation in pressure is used to calculate systolic and diastolic pressure, artery lumen area compliance and artery volume compliance, artery lumen area, and the blood flow rate through the patient's arteries (e.g. the brachial artery for the case of the patient's arm, or the femoral artery or the case of the patient's leg).

Drzewiecki, et al. (U.S. Pat. No. 6,338,719) appears to describe a method and system detecting various vascular conditions using an occlusive arm cuff plethysmograph. The system includes data acquisition hardware, including the occlusive arm cuff plethysmograph, for obtaining arterial and endothelial function data from a patient, processing means utilizing application or analysis software for analyzing the arterial and endothelial function data, and a database of computer models, such as brachial artery pressure versus lumen area curves (P-A curves) and brachial artery pressure versus compliance curves (P-C curves), developed by analyzing data for a plurality of subjects where their vascular conditions were known. The processing means diagnoses and predicts various vascular conditions pertaining to the patient by comparing or correlating the analyzed arterial and endothelial function data with the computer models stored within the database and presents the findings on a display.

Drzewiecki, et al. (U.S. Pat. No. 6,626,840) appears to describe a method and system provided for detecting various vascular conditions using an occlusive arm cuff plethysmograph. The system includes data acquisition hardware, including the occlusive arm cuff plethysmograph, for obtaining arterial and endothelial function data from a patient, processing means utilizing application or analysis software for analyzing the arterial and endothelial function data, and a database of computer models, such as brachial artery pressure versus lumen area curves (P-A curves) and brachial artery pressure versus compliance curves (P-C curves), developed by analyzing data for a plurality of subjects where their vascular conditions were known. The processing means diagnoses and predicts various vascular conditions pertaining to the patient by comparing or correlating the analyzed arterial and endothelial function data with the computer models stored within the database and presents the findings on a display.

Chowienczyk, et al. (U.S. Pat. No. 6,908,436) appears to describe a method of measuring endothelial function in a person, where the method comprises applying pressure to one arm of the person such as to restrict blood flow in the arm, releasing the pressure in order to cause an increase in blood flow in the arm due to reactive hyperemia, and then measuring the difference in pulse propagation time between the two arms of the person.

Dafni (U.S. Pat. No. 7,390,303) appears to disclose a method and apparatus for assessment of relative changes in the cross sectional area of a limb artery. The method includes applying to the artery an external pressure, that causes the cross-sectional area of the artery to change between systole and diastole much more than if the pressure is not applied, determining, over one or more cardiac cycles, a baseline value for a parameter related to the cross-sectional area of the artery, while the pressure is applied, applying a stimulus to the artery, determining, over one or more cardiac cycles, a stimulus-affected value for the parameter related to the cross-sectional area of the artery, while the pressure is applied and while the artery is in a dilated state affected by the stimulus and evaluating the artery based on a comparison of the determined stimulus-affected and baseline values, the baseline value is determined while the artery is substantially not affected by the stimulus.

Whitt et al. (US 2010/0305459 A1) described a method for measuring reactive hyperemia in a subject. The method includes performing a first segmental cuff plethysmography to generate a baseline arterial compliance curve and/or a baseline pressure-area (P-A) curve, performing a second segmental cuff plethysmography to generate a hyperemic arterial compliance curve and/or a hyperemic P-A curve, and calculating an area between the baseline and the hyperemic curves. The size of the area can be used as an indication of endothelial dysfunction (ED) and ED-related diseases.

Raines et al. (U.S. Pat. No. 6,152,881) describes a calibrated method for characterizing blood flow in a limb of a patient during reactive hyperemia. The method establishes a predetermined, near diastolic, pressure in said blood pressure cuff during the reactive hyperemic episode, continually senses the pressure in the cuff and periodically changes the internal volume of said blood pressure cuff by a predetermined volumetric amount to calibrate the system. The resultant change in the pressure is a calibration pressure pulse and is used to calculate pulsatile blood volume through the blood vessel.

It is believed the methods and apparatus described herein offer significant advantages over aforementioned methods.

### SUMMARY

Conventional flow mediated dilation (FMD) studies measure arterial diameter before and after the application of an endothelial stimulus. In the methods and apparatus described herein the cross sectional area of the artery rather than the diameter is measured. Rather than employing B-mode ultrasound to image the arterial lumen, a simple inexpensive blood pressure cuff is utilized.

Accordingly, in certain embodiments, a method of assessing endothelial function in a mammal, is provided where the method comprises applying to the artery a substantially constant external pressure that causes the tension in the artery wall to decrease, where the pressure is provided via a cuff adjacent to and/or around a region of the mammal's body; determining, over the course of one or more cardiac cycles, changes in pressure in the cuff resulting from cardiac activity of the mammal, or an artificially induced arterial pulse to establish a baseline value for a parameter related to endothelial function in the mammal; applying a stimulus to the mammal; and determining, over the course of one or more cardiac cycles, changes in pressure in the cuff resulting from cardiac activity of the mammal, or an artificially induced arterial pulse, to determine a stimulus-effected value for a parameter related to endothelial function in the mammal; where the baseline value is determined from measurements made when the mammal is not substantially effected by the stimulus; and where differences in the baseline value and the stimulus-effected value provide a measure of endothelial function in the mammal. In certain embodiments establishing a baseline value comprises establishing a baseline value for an artificially induced arterial pulse. In certain embodiments determining a stimulus-effected value comprises determining a stimulus-effected value for an artificially induced arterial pulse. In certain embodiments establishing a baseline value comprises establishing a baseline value for changes in pressure resulting from cardiac activity of the mammal. In certain embodiments determining a stimulus-effected value comprises establishing a stimulus-effected value for changes in pressure resulting from cardiac activity of the mammal. In certain embodiments the substantially constant pressure is applied by a pressurized cuff disposed around an arm or leg of the mammal. In certain embodiments the cuff is pressurized by a gas or gas mixture or by a liquid or gel. In certain embodiments the substantially constant external pressure is maintained by a system (e.g., a controller comprising circuitry) that monitors and adjusts the pressure in the cuff and whose response time is sufficiently slow so that the changes in pressure resulting from the cardiac activity are not substantially attenuated by the system. In certain embodiments the response time is sufficiently slow so that the pressure changes resulting from the cardiac activity are attenuated by less than about 20%, more preferably less than about 15%, still more preferably less than about 10%, or less than about 5%. In certain embodiments the substantially constant external pressure is maintained by setting the pressure in the cuff to a value and not altering external pressure applied to the cuff during the measurements of pressure variations due to the cardiac activity. (In such embodiments, pressure control can comprise simple “on/off” control. In certain embodiments applying the pressure to the artery comprises applying a local pressure that does not substantially affect other blood vessels in a same limb as the artery. In certain embodiments applying the external pressure to the artery comprises applying a pressure that affects an entire cross-section of a limb including the artery. In certain embodiments the substantially constant external pressure is equivalent to or below the average diastolic pressure measured for the subject. In certain embodiments the substantially constant external pressure is below the average diastolic pressure measured for the subject or below an expected diastolic pressure for the subject. In certain embodiments the substantially constant external pressure is below the average diastolic pressure measured for the subject, but is no more than about 5 mm Hg below the average diastolic pressure, or no more than about 10 mm Hg below the average diastolic pressure. In certain embodiments the substantially constant external pressure is set to a predetermined pressure. In certain embodiments the substantially constant pressure is set at different levels during measurement phases. In certain embodiments the baseline value is determined before applying the stimulus. In certain embodiments the baseline value is determined after applying the stimulus. In certain embodiments the determining, over the course of one or more cardiac cycles, changes in pressure in the cuff resulting from cardiac activity of the mammal comprises determining the pressure in the cuff as a function of time. In certain embodiments the determining comprises integrating the value of a pressure change over time (calculating the area under a pressure/time curve) for one or for a plurality (e.g., as least 2 cycles, or at least 5 cycles, or at least 10 cycles, or at least 15 cycles, or at least 20 cycles or at least 25 cycles, or at least 30 cycles or at least 50 cycles, or at least 75 cycles, or at least 100 cycles) to determine an integrated pressure value. In certain embodiments the determining comprises determining the maximum, or a certain percentile rank of the derivative of the pressure versus time wave form on the rising edge of a pressure pulse for one or for a plurality of cardiac cycles to determine a compliance value. In certain embodiments the integrated pressure value and/or the compliance value is averaged over a plurality of cardiac cycles (e.g., as least 2 cycles, or at least 5 cycles, or at least 10 cycles, or at least 15 cycles, or at least 20 cycles or at least 25 cycles, or at least 30 cycles or at least 50 cycles, or at least 75 cycles, or at least 100 cycles) or the integrated pressure value and/or the compliance value is determined for a single cardiac cycle. In certain embodiments the single cardiac cycle is a cardiac cycle selected for the maximum change in the value in a plurality of cardiac cycles. In certain embodiments the single cardiac cycle is a cardiac cycle selected for the maximum change in the value between a baseline measurement and a stimulus-effected measurement. In certain embodiments applying the stimulus comprises restricting flow of blood to the limb by occlusion of a blood vessel (e.g., using a cuff and/or a tourniquet). In certain embodiments restricting the flow of blood and applying the pressure on the artery are performed using separate cuffs. In certain embodiments the same cuff is used to occlude the blood vessel and to apply the pressure on the artery. In certain embodiments restricting flow of blood through the artery comprises inflating the restricting cuff to a pressure at least 10 mm Hg above measured systolic blood pressure for the mammal. In certain embodiments restricting flow of blood through the artery comprises inflating the restricting cuff to a predetermined pressure. In certain embodiments restricting flow of blood through the artery comprises restricting for at least about 30 seconds, preferably for at least about 1 minute, or at least about 90 seconds, or at least about 2 minutes. In certain embodiments applying the stimulus does not comprise restricting flow of blood to the limb by occlusion a blood vessel. In certain embodiments applying the stimulus comprises administering a drug to the patient. In certain embodiments the drug is not an NO agonist. In certain embodiments the drug is a β₂-adrenergic agonist. In certain embodiments the drug is an NO donor (e.g., nitroglycerin, sodium nitroprusside, etc.). In certain embodiments the stimulus does not comprise occlusion of an artery and/or does not comprise administration of a drug. In certain embodiments the stimulus comprises low intensity ultrasound and/or acoustic/mechanical tissue vibration.

In various embodiments methods of assessing endothelial function in a mammal are provided where the methods comprise applying to the artery at a first location a substantially constant external pressure that causes the artery to fully or partially collapse, where the pressure is provided via a first cuff adjacent to and/or around a region of the mammal's body; applying to the artery at a second location a substantially constant external pressure that causes the artery to fully or partially collapse, where the pressure is provided via a second cuff adjacent to and/or around a region of the mammal's body; determining, over the course of one or more cardiac cycles, changes in pressure in the first cuff resulting from cardiac activity of the mammal or an artificially induced arterial pulse, determining, over the course of one or more cardiac cycles, changes in pressure in the second cuff resulting from cardiac activity of the mammal or an artificially induced arterial pulse; and calculating a baseline transit time of a pressure pulse from the first cuff to the second cuff to establish a baseline value for a parameter related to endothelial function in the mammal; applying a stimulus to the mammal; determining, over the course of one or more cardiac cycles, changes in pressure in the first cuff resulting from cardiac activity of the mammal or an artificially induced arterial pulse, determining, over the course of one or more cardiac cycles, changes in pressure in the second cuff resulting from cardiac activity of the mammal or an artificially induced arterial pulse; and calculating the stimulus-effected transit time of a pressure pulse from the first cuff to the second cuff to establish a stimulus-effected value for a parameter related to endothelial function in the mammal; where the baseline value is determined from measurements made when the mammal is not substantially effected by the stimulus; and where differences in the baseline value and the stimulus-effected value for the transit time provide a measure of endothelial function in the mammal. In certain embodiments the baseline transit time is calculated for an artificially induced arterial pulse. In certain embodiments the stimulus-effected transit time is calculated for an artificially induced arterial pulse. In certain embodiments the baseline transit time and/or the stimulus-effected transit time is calculated for a pulse resulting from cardiac activity in the mammal. In certain embodiments the first cuff and the second cuff are disposed around an arm or leg of the mammal. In certain embodiments the first cuff and the second cuff are pressurized by a gas or gas mixture or by a liquid or gel. In certain embodiments the first cuff and the second cuff are maintained at substantially the same substantially constant pressure. In certain embodiments the first cuff and the second cuff are maintained at different substantially constant pressures. In certain embodiments the substantially constant external pressure(s) are maintained by a system that monitors and adjusts the pressure in the first cuff and/or the second cuff and whose response time is sufficiently slow so that the changes in pressure resulting from the cardiac activity are not substantially attenuated by the system. In certain embodiments the response time is sufficiently slow so that the pressure changes resulting from the cardiac activity are attenuated by less than about 20%, more preferably less than about 15%, still more preferably less than about 10%, or less than about 5%. In certain embodiments the substantially constant external pressure is maintained by setting the pressure in the first cuff and/or the second cuff to a value and not altering external pressure applied to the first cuff and/or the second cuff during the measurements of pressure variations due to the cardiac activity. In certain embodiments applying the pressure to the artery comprises applying a local pressure that does not substantially affect other blood vessels in a same limb as the artery. In certain embodiments applying the external pressure to the artery comprises applying a pressure that affects an entire cross-section of a limb including the artery. In certain embodiments the substantially constant external pressure is equivalent to or below the average diastolic pressure measured for the subject. In certain embodiments the substantially constant external pressure is below the average diastolic pressure measured for the subject or below an expected diastolic pressure for the subject. In certain embodiments the substantially constant external pressure is below the average diastolic pressure measured for the subject, but is no more than about 10 mm Hg below the average diastolic pressure. In certain embodiments the substantially constant external pressure is set to a predetermined pressure. In certain embodiments the substantially constant pressure is set at different levels during measurement phases. In certain embodiments the baseline value is determined before applying the stimulus or after applying the stimulus. In certain embodiments the determining, over the course of one or more cardiac cycles, changes in pressure in the first cuff and/or the second cuff resulting from cardiac activity of the mammal comprises determining the pressure in the first and/or second cuff as a function of time. In certain embodiments determining the transit time comprises comparing the changes in pressure as a function in time in the first cuff to the changes in pressure as a function of time in the second cuff and identifying the one or more corresponding pressure pulses in the first cuff and in the second cuff and determining the delay between the occurrence of one or of a plurality of pressure pulses in the first cuff and the occurrence of one or of a plurality of corresponding pressure pulses in the second cuff to calculate a transit time for one or more pressure pulses. In certain embodiments the transit time is averaged over a plurality of cardiac cycles (e.g., as least 2 cycles, or at least 5 cycles, or at least 10 cycles, or at least 15 cycles, or at least 20 cycles or at least 25 cycles, or at least 30 cycles or at least 50 cycles, or at least 75 cycles, or at least 100 cycles). In certain embodiments the transit time is determined for a single cardiac cycle. In certain embodiments the single cardiac cycle is a cardiac cycle selected for the maximum change in transit time between a baseline measurement and a stimulus-effected measurement. In certain embodiments applying the stimulus comprises restricting flow of blood to the limb by occlusion a blood vessel. In certain embodiments restricting the flow of blood is accomplished using a cuff and/or a tourniquet. In certain embodiments restricting the flow of blood and applying the pressure on the artery are performed using a cuff other than the first or second cuff. In certain embodiments the first cuff is used to occlude the blood vessel. In certain embodiments restricting flow of blood through the artery comprises inflating the restricting cuff to a pressure at least about 10 mm Hg above measured systolic blood pressure for the mammal. In certain embodiments restricting flow of blood through the artery comprises inflating the restricting cuff to a predetermined pressure. In certain embodiments restricting the flow of blood through the artery comprises restricting for at least about 30 seconds, preferably for at least about 1 minute, or at least about 90 seconds, or at least about 2 minutes. In certain embodiments applying the stimulus does not comprise restricting flow of blood to the limb by occlusion a blood vessel. In certain embodiments applying the stimulus comprises administering a drug to the patient. In certain embodiments the drug is not an NO agonist. In certain embodiments the drug is a β₂-adrenergic agonist. In certain embodiments the drug is an NO donor (e.g., nitroglycerin, sodium nitroprusside, etc.). In certain embodiments the stimulus does not comprise occlusion of an artery and/or does not comprise administration of a drug. In certain embodiments the stimulus comprises low intensity ultrasound and/or acoustic/mechanical tissue vibration.

In various embodiments an apparatus for assessment endothelial function in a mammal is provided where the apparatus comprises a measurement cuff adapted to apply a substantially constant pressure to an artery in the mammal; a measurement unit adapted to detect and quantify over one or more cardiac cycles, pressure pulses in the cuff while the substantially constant pressure is applied; a controller that is adapted to apply to the cuff a said substantially constant pressure where said controller monitors and adjusts the pressure in said cuff and whose response time is sufficient slow so that the changes in pressure resulting from said cardiac cycles are not substantially attenuated by said system, and/or that is adapted to control a pressure source and a valve to provide on-off control of the pressure in said cuff; and a processor adapted to analyze, and/or store, and/or compare values determined from the pressure pulses in at least two measurement rounds. In certain embodiments the measurement unit and controller are combined into an integrated control unit. In certain embodiments the measurement unit, controller, and processor are integrated into an integrated control unit. In certain embodiments the controller (or integrated control unit) is configured to monitor and adjust the substantially constant pressure at a response time sufficiently slow so that the pressure changes resulting from the cardiac activity are attenuated by less than about 20%, more preferably less than about 15%, still more preferably less than about 10%, or less than about 5%. In certain embodiments the controller (or integrated control unit) is configured to maintain the substantially constant external pressure by setting the pressure in the cuff to a value and not altering external pressure applied to the cuff during the measurements of pressure variations due to the cardiac activity. In certain embodiments the controller (or integrated control unit) is configured to apply a substantially constant external pressure equivalent to or below a diastolic pressure determined for the subject. In certain embodiments the controller (or integrated control unit) is configured to apply a substantially constant external pressure below the average diastolic pressure measured for the subject or below an expected diastolic pressure for the subject. In certain embodiments the controller (or integrated control unit) is configured to apply a substantially constant external pressure below the average diastolic pressure measured for the mammal, but no more than about 10 mm Hg below the average diastolic pressure. In certain embodiments the controller (or integrated control unit) is configured to apply the substantially constant pressure at different levels during measurement phases. In certain embodiments the measurement apparatus comprises a pump (e.g., a hydraulic pump, a pneumatic pump, etc.) adapted to apply the pressure to the cuff. In certain embodiments the response time is reduced by disposing a narrow pressure line between hydraulic or pneumatic pump and the cuff. In certain embodiments the apparatus comprises a valve and a pump configured to provide on-off control of the pressure in the cuff. In certain embodiments the apparatus further comprises an accelerometer disposed to detect movement or vibrations in the cuff or apparatus. In certain embodiments the cuff is pressurized with a material selected from the group consisting of a gas, a fluid, and a gel. In certain embodiments the cuff is adapted to apply pressure substantially around an entire circumference of a limb including the artery. In certain embodiments the cuff is adapted to apply a local pressure that does not substantially affect other blood vessels in a same limb as the artery. In certain embodiments the processor is configured to determine a blood pressure. In certain embodiments the processor is configured to calculate the substantially constant pressure based on one or more blood pressure measurements and to direct the controller to apply the calculated substantially constant pressure. In certain embodiments the processor is configured to determine comparing pulse properties such as amplitude and maximum upward slope, before and after stimulus, and to determine if pulses were measured at similar cuff pressures and if not to correct the data for the difference in cuff pressures. In certain embodiments the controller is configured to induce at least one of measurement round responsive to an indication that a stimulus was administered to the artery and at least one of the measurement rounds before the indication that the stimulus was administered to the artery is received. In certain embodiments the controller is adapted to apply the pressure continuously over at least five cardiac cycles or at least 10 cycles, or at least 15 cycles, or at least 20 cycles or at least 25 cycles, or at least 30 cycles or at least 50 cycles, or at least 75 cycles, or at least 100 cardiac cycles of the subject. In certain embodiments the controller is configured to store over the course of one or more cardiac cycles, changes in pressure in the cuff resulting from cardiac activity of the mammal as a function of time. In certain embodiments the processor is configured to integrate the value of a pressure change over time (calculate the area under a pressure/time curve) for one or for a plurality of cardiac cycles to determine an integrated pressure value. In certain embodiments the processor is configured to determine the maximum of the derivative of the pressure versus time wave form on the rising edge of a pressure pulse for one or for a plurality of cardiac cycles to determine a compliance value. In certain embodiments the processor is configured to average the integrated pressure value and/or the compliance value over a plurality of cardiac cycles (e.g., as least 2 cycles, or at least 5 cycles, or at least 10 cycles, or at least 15 cycles, or at least 20 cycles or at least 25 cycles, or at least 30 cycles or at least 50 cycles, or at least 75 cycles, or at least 100 cycles). In certain embodiments the processor is configured to determine the integrated pressure value and/or the compliance value a single cardiac cycle. In certain embodiments the processor is configured to determine the integrated pressure value and/or the compliance value and identify a maximum change in the value between a baseline measurement and a stimulus-effected measurement.

In certain embodiments an apparatus for assessment endothelial function in a mammal is provided where the apparatus comprises a first measurement cuff adapted to apply a substantially constant first pressure to an artery in the mammal; a second measurement cuff adapted to apply a substantially constant second pressure to an artery in the mammal; a measurement unit adapted to detect and quantify over one or more cardiac cycles, pressure pulses in the first cuff and/or the second cuff while the substantially constant pressure is applied; a controller adapted to apply to the first cuff the substantially constant first pressure and/or the second cuff the substantially constant second pressure where the controller monitors and adjusts the pressure in the first cuff and/or the second cuff and whose response time is sufficient slow so that the changes in pressure in the first cuff and the second cuff resulting from the cardiac cycles are not substantially attenuated by the system; and a processor configured to monitor pressure pulses in the first cuff and in the second cuff, identify corresponding pulses in the first and the second cuff and calculate a transit time for the pressure pulse in an artery from the location of the first cuff to the second cuff. In certain embodiments the measurement unit and controller are combined into an integrated control unit. In certain embodiments the measurement unit, controller, and processor are integrated into an integrated control unit. In certain embodiments the processor is configured to analyze, and/or store, and/or compare transit time values determined from the pressure pulses in at least two measurement rounds. In certain embodiments the controller (or integrated control unit) is configured to monitor and adjust the substantially constant first pressure and/or the substantially constant second pressure at a response time sufficiently slow so that the pressure changes resulting from the cardiac activity are attenuated by less than about 20%, more preferably less than about 15%, still more preferably less than about 10%, or less than about 5%. In certain embodiments the controller (or integrated control unit) is configured to maintain the substantially constant first pressure and/or the substantially constant second pressure by setting the pressure in the first cuff and/or the second cuff to a value and not altering external pressure applied to the first cuff and/or the second cuff during the measurements of pressure variations due to the cardiac activity. In certain embodiments the controller (or integrated control unit) is configured to apply a substantially constant external pressure equivalent to or below a diastolic pressure determined for the subject. In certain embodiments the controller (or integrated control unit) is configured to apply a substantially constant external pressure below the average diastolic pressure measured for the subject or below an expected diastolic pressure for the subject. In certain embodiments the controller (or integrated control unit) is configured to apply a substantially constant external pressure below the average diastolic pressure measured for the mammal, but no more than about 10 mm Hg below the average diastolic pressure. In certain embodiments the controller (or integrated control unit) is configured to apply the substantially constant pressure at different levels during measurement phases. In certain embodiments the controller (or integrated control unit) is configured to apply the same substantially constant pressure to the first cuff and to the second cuff. In certain embodiments the measurement apparatus comprises a first pump (e.g., a hydraulic pump, a pneumatic pump, etc.) adapted to apply the pressure to the first cuff and the second cuff, or a first pump (e.g., a hydraulic pump, a pneumatic pump, etc.) adapted to apply the pressure to the first cuff and a second pump (e.g., a hydraulic pump, a pneumatic pump, etc.) adapted to apply the pressure to the second cuff. In certain embodiments the response time is reduced by disposing a narrow pressure line between the first pump and the first and second cuff, or between the first pump and the first cuff and the second pump and the second cuff. In certain embodiments the apparatus comprises a valve and a pump configured to provide on-off control of the pressure in the cuff. In certain embodiments the apparatus further comprises an accelerometer disposed to detect movement or vibrations in the cuff or apparatus. In certain embodiments the first cuff and the second cuff are pressurized with a material selected from the group consisting of a gas, a fluid, and a gel. In certain embodiments the first cuff and the second cuff are adapted to apply pressure substantially around an entire circumference of a limb including the artery. In certain embodiments the first cuff and the second cuff are adapted to apply a local pressure that does not substantially affect other blood vessels in a same limb as the artery. In certain embodiments the processor is configured to determine a blood pressure. In certain embodiments the processor is configured to calculate the substantially constant pressure based on one or more blood pressure measurements and to direct the controller to apply the calculated substantially constant pressure. In certain embodiments the processor is configured to determine comparing pulse properties such as amplitude and maximum upward slope, before and after stimulus, and to determine if pulses were measured at similar cuff pressures and if not to correct the data for the difference in cuff pressures. In certain embodiments the controller is configured to induce at least one of measurement round responsive to an indication that a stimulus was administered to the artery and at least one of the measurement rounds before the indication that the stimulus was administered to the artery is received. In certain embodiments the controller is adapted to apply the pressure continuously over at least five cardiac cycles or at least 10 cycles, or at least 15 cycles, or at least 20 cycles or at least 25 cycles, or at least 30 cycles or at least 50 cycles, or at least 75 cycles, or at least 100 cardiac cycles of the subject. In certain embodiments the controller is configured to store over the course of one or more cardiac cycles, changes in pressure in the cuff resulting from cardiac activity of the mammal as a function of time. In certain embodiments the processor is configured to integrate the value of a pressure change over time (calculate the area under a pressure/time curve) for one or for a plurality of cardiac cycles (e.g., as least 2 cycles, or at least 5 cycles, or at least 10 cycles, or at least 15 cycles, or at least 20 cycles or at least 25 cycles, or at least 30 cycles or at least 50 cycles, or at least 75 cycles, or at least 100 cycles) to determine an integrated pressure value. In certain embodiments the processor is configured to determine the maximum of the derivative of the pressure versus time wave form on the rising edge of a pressure pulse for one or for a plurality of cardiac cycles to determine a compliance value. In certain embodiments the processor is configured to average the integrated pressure value and/or the compliance value over a plurality of cardiac cycles. In certain embodiments the processor is configured to determine the integrated pressure value and/or the compliance value a single cardiac cycle. In certain embodiments the processor is configured to determine the integrated pressure value and/or the compliance value and identify a maximum change in the value between a baseline measurement and a stimulus-effected measurement.

### BRIEF DESCRIPTION OF THE DRAWINGS

FIG. 1 provides a schematic illustration of a system 100 for assessing arterial endothelial function in a mammal.

FIG. 2 shows distension of the brachial artery recorded by M-mode wall tracking Top: Distension waveform under normal conditions. Bottom: When the transmural pressure is decreased by 80 mmHg using an external cuff, the maximum distention of the artery increases more than twenty-fold over baseline conditions.

FIG. 3 shows typical single pulse waveforms obtained from the pressure cuff when inflated to 70 mmHg. Both the amplitude and slope of the rising edge of the pulse increase markedly after endothelial stimulation. This individual thus exhibits intact endothelial response. Administration of the NO synthase inhibitor L-NAME greatly attenuates this response, suggesting that the measurement is primarily sensitive to NO-mediated vasodilation.

FIG. 4 shows results of a study of the effects of a five minute cuff occlusion on the area (upper panel) and maximum derivative of the area vs. time curve (lower panel). Both quantities increase markedly after cuff release but have returned to baseline levels after 25 minutes.

FIG. 5 shows results of a study of the effects of two serial five minute cuff occlusions on the area (upper panel) and maximum derivative of the area vs. time curve (lower panel). Both quantities increase markedly after cuff release but have returned to baseline levels after approximately 10 minutes.

FIG. 6 shows results of a study of the same individual in which the same protocol is performed except that the cuff is not inflated to suprasystolic levels. Some natural drift in the baseline signals is evident, but the magnitude of this variation is far less than the response elicited by reactive hyperemia.

FIG. 7 provides a block diagram of a control 200 (111) in accordance with one illustrative embodiment of the present invention.

FIG. 8 provides a schematic view of one embodiment of a pneumatic/hydraulic unit 214 shown in FIG. 7.

FIG. 9 provides flow chart illustrating typical acts performed in a measurement of the effect of a stimulus on endothelial function.

FIG. 10 shows responses to 0.4 mg and 0 mg sublingual NG in one individual. The upper figure shows relative change in area, while the lower figure shows relative change in compliance. Measurements are shown ±SEM.

FIG. 11 shows responses to RH after a five-minute cuff occlusion in a 28 year-old male. The upper figure shows relative change in area, while the lower figure shows relative change in compliance. Measurements are shown ±SEM.

FIG. 12 shows the fractional change in pulse amplitude (proportional to area) observed relative to baseline for all studies analyzed. It is clear that the method detects much larger changes in the cases where RH or NG is used as stimulus than when no stimulus is applied. The fact that there is totally unambiguous distinction between the stimulus-present versus NS studies in all cases for the time points in the range of 8-10 minutes is extremely encouraging.

FIG. 13 illustrates one embodiment of a system that uses an on-off control system to set the cuff pressure to a constant value during measurement. This is effected by a microcontroller that actuates a pump and a valve.

FIG. 14 shows a photograph of a portable prototype device (top) and close-up (bottom).

FIG. 15 shows the typical decrease in cuff pressure during the measurement interval owing to the displacement of tissue under the cuff. The analysis method preferably takes this characteristic into account.

FIG. 16 illustrates the change in arterial compliance with transmural pressure (blood pressure minus cuff pressure). These data were obtained using intraarterial ultrasound and blood pressure measurement.

### DETAILED DESCRIPTION

In various embodiments, methods and devices are provided for non-invasively assessing arterial endothelial function in a mammal (e.g., a human or a non-human mammal), particularly in response to a stimulus. The change in endothelial function (or lack of change) in response to particular stimuli provides a measure of the vascular health of the subject.

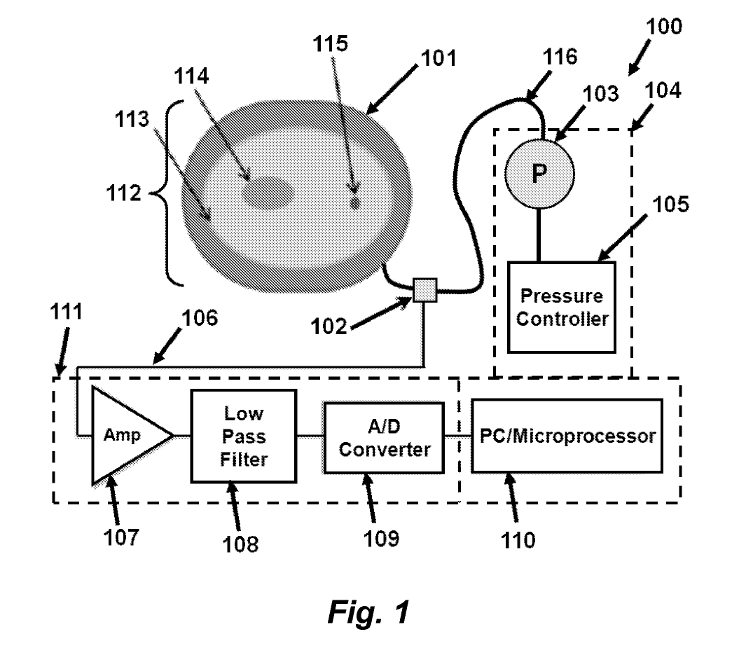

Consider FIG. 1 which provides a schematic representation of the cross section of the human upper arm 112 enclosed in an inflated blood pressure cuff 101. In conventional blood pressure measurement, the cuff is initially inflated above the systolic blood pressure. This applies pressure to the skin surface 113 which compresses the arm and the contents thereof (e.g., humerus 114, brachial artery 115, etc.) causing the underlying arteries 115 to collapse. The pressure in the cuff in this case is purely determined by the external pressure applied by the air in the cuff.

Consider the case where the cuff is inflated to a pressure below diastolic pressure. This distorts the shape of the artery causing the artery to partially collapse. As the pressure in the artery increases during the course of the natural blood pressure pulse (i.e., exceeds the diastolic pressure), the flattened artery expands. As a consequence of the near incompressibility of human tissue and body fluids, the pressure in the cuff increases in proportion to the increase in arterial cross sectional area. By measuring the pressure in the cuff, it is thus possible to obtain a measure of arterial caliber.

Consider an illustrative example, where 70 mmHg pressure is applied to the cuff when the subject's diastolic pressure is 80 mmHg. In certain embodiments this is accomplished by attaching a constant pressure source 103 to the cuff that provides the 70 mm Hg pressure. In various embodiments the constant pressure source 103 utilizes a hydraulic or pneumatic pump or pressurized gas, or a fluid reservoir. Such sources typically utilize a servo/valve mechanism to maintain the pressure set point, and this servo can be under control of a pressure controller 105. In some embodiments, a pump and valve are actuated by a control system in order to keep the pressure within an acceptable range (e.g. ±5 mmHg) about the set point.

To preserve pressure signals resulting from cardiac activity (i.e., cardiac cycle(s)) it is desirable that the pressure source not substantially cancel out the changes in cuff pressure due to the increase in area of the flattened vessel. This may be achieved by increasing the time constant of the system response of the servo/pressure controller system and/or more simply by placing a flow resistor 116 between the pressure source and the cuff. In the simplest implementation, a long thin tube (e.g., 1 m (or other) length of thin intervening tubing that serves as a pneumatic low pass filter) can provide this resistance. Another option is to decouple the constant pressure source from the cuff once the cuff has reached its target pressure.

In various illustrative embodiments, the time constant of the pressure system is sufficiently slow relative to pressure changes introduced by the cardiac cycle that pressure changes resulting from cardiac activity (e.g., pulse-associated pressure changes) are attenuated by less than 20%, or less than about 15%, or less than about 10%, or less than about 5%, or less than about 1% of the maximum pressure change. Similarly a substantially constant pressure is a pressure that when averaged over a sufficiently long time period that pulse-induced pressure changes are averaged out, the average pressure applied to the cuff over the desired time period various by less than 20%, more preferably less than about 15%, or less than about 10%, most preferably less than about 5%, 3%, 2%, or 1% of the applied pressure.

In various embodiments the pressure in the cuff is measured using a pressure transducer (pressure sensor) 102. One illustrative suitable pressure sensor is the Millar catheter pressure sensor (Mikro-tip, Millar Instruments, Houston, Tex.) but most low cost constitute suitable transducers. The output signal of transducer can be amplified (e.g., using an instrumentation amplifier such as AD627, Analog Devices, Inc., Norwood Mass.), optionally low-pass filtered (e.g., using 8th Order elliptic Filter, LTC-1069-6, Linear Technology Corp., Milpitas, Calif.), and then digitized (e.g., at 1 kHz using a A/D converter PCI card (NI-6035, National Instruments, Austin, Tex.).

The digitized signal can be directly interpreted as a quantity proportional to the area of the arterial lumen as long as the pressure in the cuff is less than the systolic pressure of the subject, and as long as the pressure at the outlet of the pressure source is held substantially constant. The pressure source we used in one prototype (Hokanson E20, Bellevue, Wash.) provides servo regulation that is too fast to allow its direct application to the cuff without attenuating the signal due to the expansion of the arterial lumen. Consequently, we employed a 1 m length of thin intervening tubing to serves as a pneumatic low pass filter.

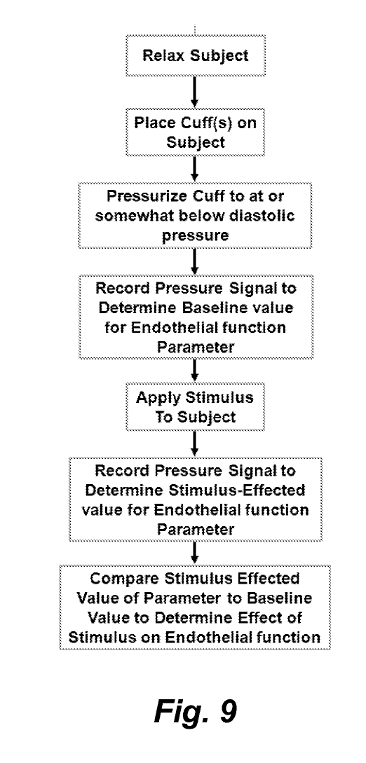

An illustrative, but non-limiting, protocol can involve the following steps (see also flow chart in FIG. 9):

1. The subject is seated or lies supine and rests briefly, e.g., for five minutes.

2. The subject's blood pressure is measured.

3. The cuff is inflated to at or, preferably somewhat below, the diastolic pressure (e.g., 10 mmHg below the diastolic blood pressure) and the pressure signal is recorded to determine a baseline value for a parameter related to endothelial function in said mammal (e.g. integrated pressure as a function of time).

4. A stimulus is applied to the subject.

5. A pressure signal is recorded with the cuff inflated to at or, preferably somewhat below, the diastolic pressure (e.g., 10 mmHg below the diastolic blood pressure) and the pressure signal is recorded to determine a stimulus-effected value for a parameter related to endothelial function in said mammal (e.g. integrated pressure as a function of time).

6. The stimulus-effected value of the parameter is compared to the baseline value of the parameter to determine presence, absence, and/or degree of endothelial response to said stimulus.

Any of a number of different types of stimuli can be used. Typically, however, the stimulus is one expected to have an effect on endothelial function in a mammal. Such stimuli include, but are not limited to occlusion of blood flow, application of drugs (e.g., NO agonists, β₂-adrenergic agonists such as albuterol, acoustic/mechanical tissue vibration, ultrasound stimulus, and the like).

One illustrative non-limiting protocol where the stimulus comprises occlusion of blood flow can involve the following steps:

1. Subject is seated or lies supine and rests for five minutes.

2. The subject's blood pressure is measured.

3. The cuff is inflated to 10 mmHg below the diastolic blood pressure for one minute. During this time, the pressure signal is recorded to determine a baseline value for a parameter related to endothelial function in said mammal.

4. The cuff is deflated for 30 seconds to allow blood flow to return to normal.

5. The cuff is inflated to 40 mmHg above systolic pressure for five minutes.

6. The cuff is released for 35 seconds to allow reactive hyperemia to ensue.

7. The cuff is inflated to 10 mmHg below the diastolic blood pressure for three minutes. During this time, the pressure signal is recorded to determine a stimulus-effected value for a parameter related to endothelial function in said mammal.

6. The stimulus-effected value of the parameter is compared to the baseline value of the parameter to determine presence, absence, and/or degree of endothelial response to said stimulus.

Illustrative parameters related to endothelial function is the peak value of a pressure pulse or the maximum peak value of a number of pressure pulses, or the average or median peak value of a number of pressure pulses. Other illustrative parameters include, but are not limited to the area under a pulse in a pressure versus time plot (i.e., the integrated value of pressure as a function of time) for a pulse, the peak integrated value of a series of pulses, or the average or median integrated value of a series of pulses.

Another useful parameter is the derivative of the area vs. time waveform, preferably the maximum of this derivative on the rising edge of the pulse. If the endothelial stimulus does not affect systemic systolic or diastolic blood pressure (which is a very reasonable assumption), we can assume that the pressure at the point at which the slope of the area versus time curve is maximal is approximately the same before and after endothelial stimulus. In this case, this slope is an approximately proportional to dA/dP, which is the compliance of the vessel (A and P represent area and pressure, respectively). Compliance is the fundamental quantity reduced by the smooth muscle relaxation that is a consequence of healthy endothelial response. It constitutes a extremely valuable “root cause” metric.

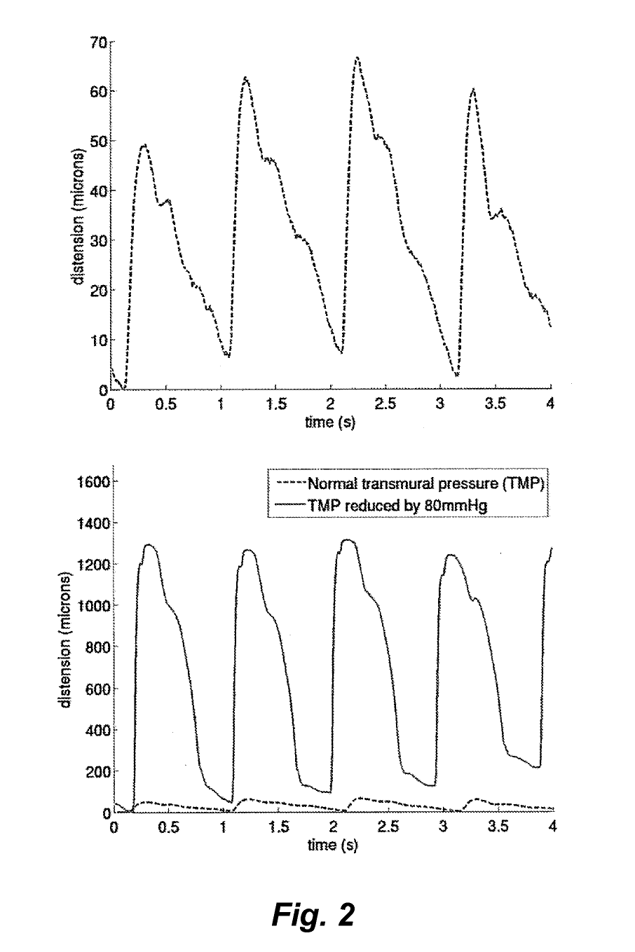

A fundamental advantage of the present methods over traditional measures of flow-mediated vasodilation (FMD) is the increased sensitivity that comes from measuring parameters related to arterial cross-sectional area rather than radius, since area is approximately proportional to the square of the radius. Also, by decreasing the transmural pressure on the artery using an external cuff inflated just below diastolic levels, the distensibility of the artery is increased by more than an order of magnitude (Bank et al. (1995) Circ. Res., 77(5): 1008-1016; Bank et al. (1999) Circulation, 100: 41-47; Kim et al. (2004) Ultrasound in Medicine & Biology, 30: 761-771). As FIG. 2 illustrates, we have observed this effect in our laboratory using M-mode ultrasound to track the arterial wall. These two factors combined lead impart exceptionally high sensitivity to the methods and devices described herein.

We have used the R-wave of the patient ECG as timing reference to facilitate the analysis of individual pulses. It is possible, however, to perform such analysis using the pressure waveform alone, which is one presently preferred method.

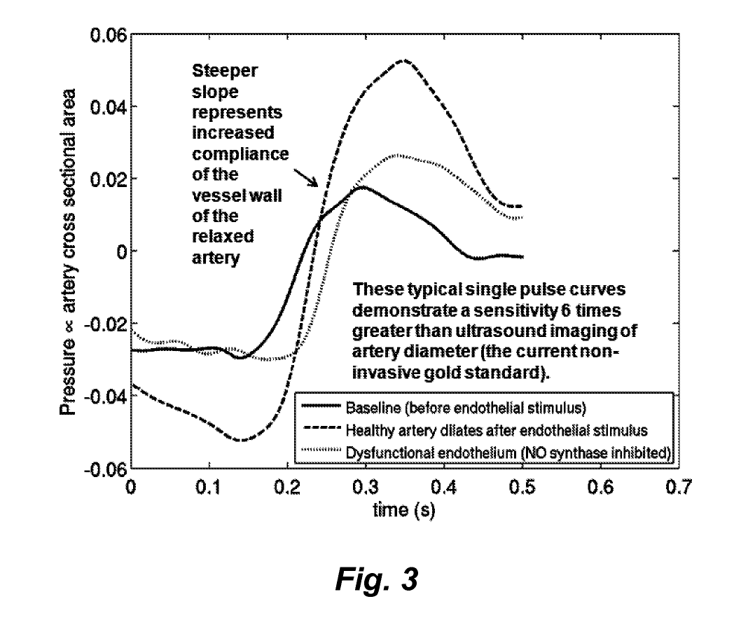

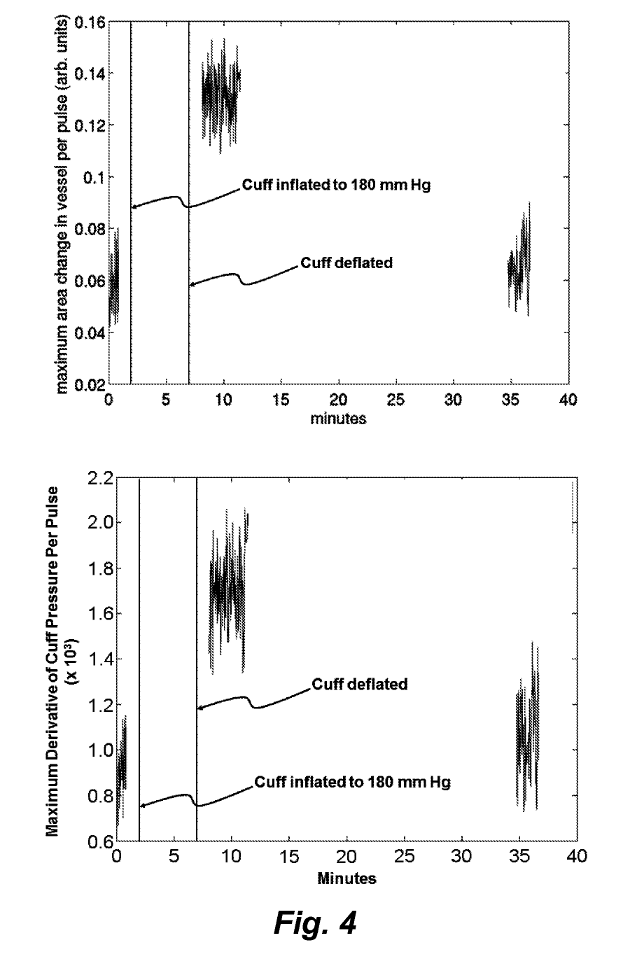

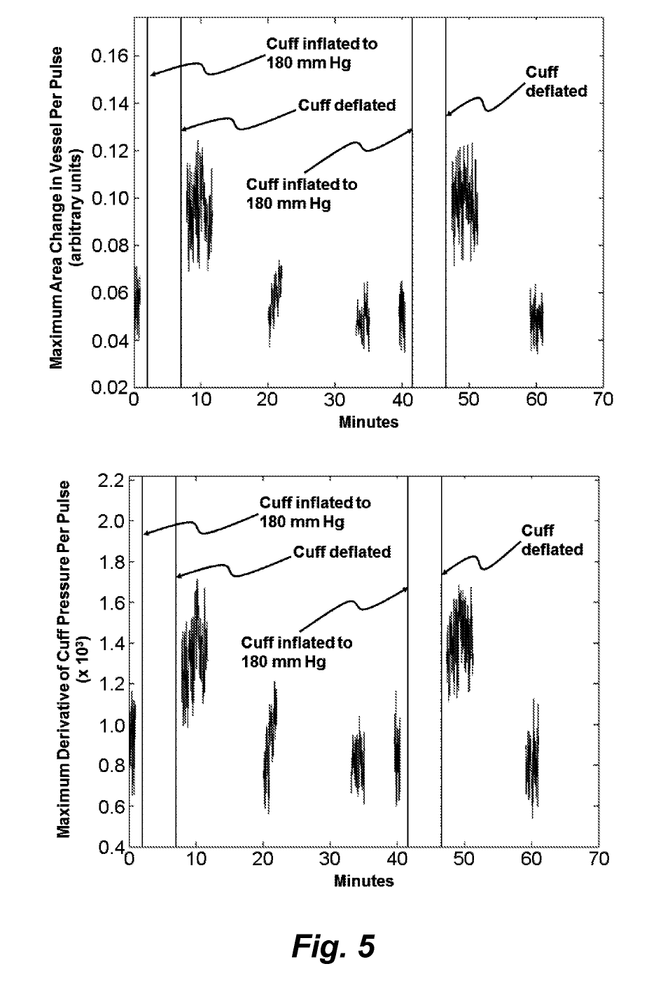

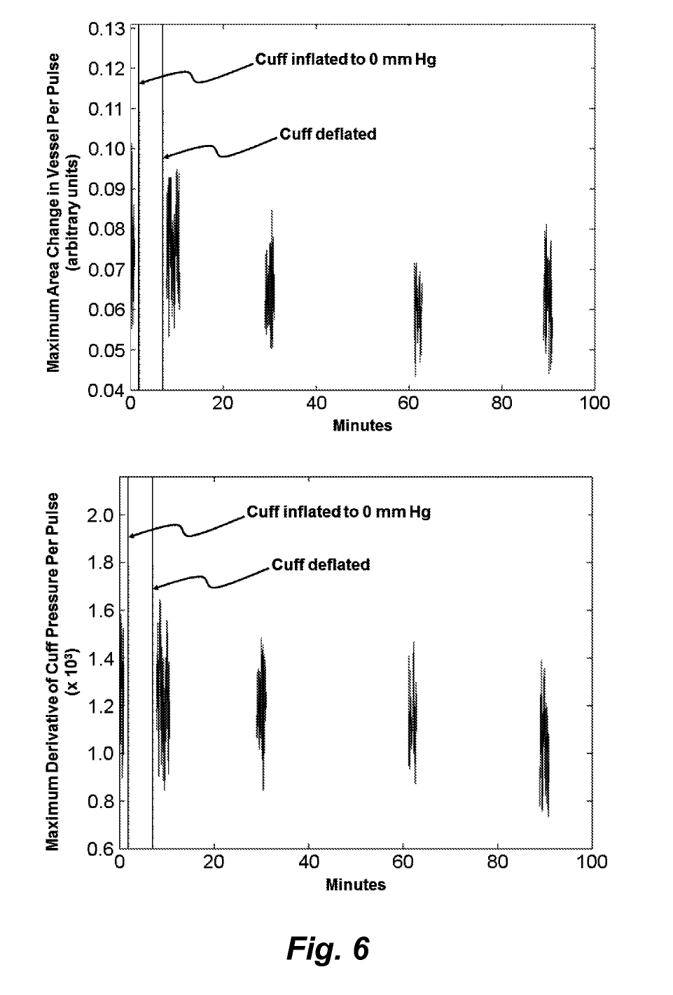

FIG. 3 shows typical single pulse waveforms obtained by measuring pressure changes in the cuff. In an artery with intact endothelial function, both pulse height (maximal cross-sectional arterial area) and compliance (maximum slope of the rising edge) increase markedly over baseline. When NO synthase is blocked via the inhibitor L-NAME, both pulse height and slope increases are greatly attenuated. FIG. 4 illustrates how a five minute cuff occlusion and the ensuring reactive hyperemia lead to major increases in area change per pulse and the maximum derivative of the area per pulse. Both metrics return to baseline levels after 20 minutes. FIG. 5 confirms the repeatability of the protocol by illustrating the effects of a series of two cuff occlusion periods. We see from FIG. 6 that only a small slow drift in the measured quantities occurs when no reactive hyperemic stimulus is applied.

In another embodiment, two cuffs are used on the same limb and inflated to some substantially constant pressure. Pressure pulses resulting from cardiac activity (cardiac cycles) are detected in each cuff. The metric of vasorelaxation used is the transit time of the pulse between the two cuffs. When the vessel is dilated, the transit time decreases. Again the transit time measurement can be initially made to establish a baseline value. The subject can be administered a stimulus, and the transit time determined again to determine a stimulus-effected transit time.

An illustrative, but non-limiting, protocol can involve the following steps:

1. The subject is seated or lies supine and rests briefly, e.g., for five minutes.

2. The subject's blood pressure is measured.

3. Both cuffs are inflated to at or, preferably somewhat below, the diastolic pressure (e.g., 10 mmHg below the diastolic blood pressure) and the pressure signal in each cuff is record to calculate a baseline transit time for a pressure pulse from the medial cuff to the distal cuff.

4. A stimulus is applied to the subject.

5. A pressure signal is recorded with both cuffs inflated to at or, preferably somewhat below, the diastolic pressure (e.g., 10 mmHg below the diastolic blood pressure) and the pressure signal in each cuff is record a stimulus-effected transit time for a pressure pulse from the medial cuff to the distal cuff.

6. The stimulus-effected value of the transit time is compared to the baseline value of the transit time to determine presence, absence, and/or degree of endothelial response to said stimulus.

In various embodiments the systems and methods described herein are suitable for ambulatory use. Inflation of the cuff, for example, can be performed using a battery powered pump, or using replaceable/refillable gas cartridges. The subject can be alerted before a scheduled measurement commences and instructed to remain still and sit or lie down.

The foregoing protocols are intended to be illustrative and not limiting. For example, while the foregoing methods are described with respect to measurement of pressure pulses in the cuff resulting from cardiac activity in the subject, they need not be sol limited. Thus, in certain embodiments, the methods involve recording artificially induced arterial pressure pulses. Methods of artificially inducing arterial pressure pulses are known to those of skill in the art. For example, Maltz and Budinger (2005) Physiol. Meas. 26: 293-307 describe the use a linear actuator to induce an artificial arterial pressure pulse. The actuator described herein employed a linear motor (from Baldor Electric Co., Fort Smith, Ark.), the actuating stem of which was adapted to make contact with the skin to introduce an artificial pulse. An applanation tonometer (SPT301, Millar Instruments, Inc., Houston, Tex.) at the free end of the stem sensed the applied force and allowed for closed-loop control of the force waveform.

In another embodiment, a cuff attached to a high bandwidth electropneumatic converter can be used to induce an artificial arterial pressure pulse. One illustrative electropneumatic converter is described by Tanaka et al. (2003) Engineering in Medicine and Biology Society, Proceedings of the 25th Annual International Conference of the IEEE, 4: 3149-3152. Tanaka et al. a disk-type cuff for local pressurization and a nozzle-flapper type electro-pneumatic converter (EPC) for the cuff-pressure control.

These embodiments are illustrative and not limiting. In view of the teachings provided herein, numerous methods to induce an artificial arterial pressure pulse are available to one of skill in the art. In certain embodiments even a standard cuff can be sufficient to induce a suitable pressure disturbance.

The systems described herein can be applied to arteries in the upper arms (or forelegs), forearms, the wrist, the thighs (hind legs), calves, ankles, and possibly even the neck (carotid arteries). In certain embodiments during the protocol, a second cuff may be applied to the contralateral limb (to which no endothelial stimulus is applied, or to which some other stimulus is applied) to serve as reference or to obtain differential measurements that elucidate the relative contributions of various vascular response mechanisms mediated by different biochemical pathways.

In various embodiments the system can be used to evaluate the effects of other stimuli including, but not limited to the influence of smooth muscle relaxation agents such as nitroglycerin, the influence of mental or physical stress, low intensity ultrasound β₂-adrenergic agonists such as albuterol, acoustic/mechanical tissue vibration, and the like. In various embodiments the cuff pressure may be set at different levels (during the measurement phase) to achieve different degrees of mechanical unloading. This can help to reduce the number of assumptions required for the interpretation of dA/dt as a measure of dA/dP. A ramping of the cuff pressure can also help to characterize the vessel more thoroughly. In various embodiments to improve signal quality, the cuff may be filled with a liquid or a gel rather than a gas.

In one particular illustrative application, the device, systems, and methods described herein are well suited for evaluation of subjects diagnosed with or at risk for sickle cell disease. In this context it is noted that the methods are highly suited to children relative to ultrasound as they are not very motion sensitive and young children are often difficult subjects. There is severe disruption of endothelial response in sickle cell disease and monitoring this can aid disease management.

FIG. 1 which provides a schematic illustration of a system 100 for assessing endothelial function in accordance with an illustrative embodiment of the methods and devices described herein. The system comprises a measurement cuff (e.g., blood pressure cuff) 112 that is configured for attachment to (around) a limb of a mammal (e.g., an arm, wrist, a leg, an ankle, etc.). The cuff can be fastened by any convenient method including, but not limited to a strap, a clip, a Velcro closure and the like. The cuff is used to administer a substantially constant pressure to the limb.

One or more bladders comprising the cuff are connected to a constant pressure source 103 that applies the constant pressure to the cuff. The pressure in the cuff in this case is purely determined by the external pressure applied by the air in the cuff. The pressure source can be coupled to a pressure controller 105 that regulates a valve or other actuator on the pressure source to regulate the substantially constant pressure applied to the cuff.

A pressure transducer (pressure sensor) 102 is disposed to monitor the pressure in the cuff. The output signal of the pressure sensor is read by a control unit 111 that comprises the circuitry necessary to read and, if necessary, to drive, the pressure sensor. In one illustrative embodiment, the control unit 111 comprises an amplifier 107 (e.g., instrumentation amplifier AD627, Analog Devices, Inc., Norwood Mass.) that amplifies the output signal of the pressure transducer, an optional low pass filter 108 (e.g., 8th Order elliptic Filter, LTC-1069-6, Linear Technology Corp., Milpitas, Calif.) and a digitizer 109 (e.g., an A/D converter PCI card (NI-6035, National Instruments, Austin, Tex.). Another tested embodiment employed a 0.6×0.6 in² MEMS pressure sensor (NPC-1210, GE Novasensor, Fremont, Calif.). The control unit 111 is configured to read the pressure from the pressure transducer.

In various embodiments the control unit 111 can be coupled to the pressure controller (e.g., via a signal cable) and thereby regulate the pressure applied to the cuff. As indicated by the dashed lines, in various embodiments, the controller 111 and pressure controller 105 can be integrated into a single control unit that both regulates the constant pressure source and reads the pressure fluctuations resulting from cardiac activity. In other embodiments, the controller 111 and pressure controller 105 can be separate units that communication (e.g., via a signal cable) or that, in certain embodiments, are independently controlled.

In certain embodiments the controller 111 as illustrated in FIG. 1, further comprises a microprocessor 110 (e.g., for signal processing and/or operating the pressure controller). The microprocessor 110 however need not be integrated into the controller, but may be a “separate” computer e.g., as described below. In certain embodiments the controller comprises a microprocessor that is itself connected to an external processor/computer. Thus, in some embodiments, the control unit may be connected to a computer via a cable for configuration and/or data download and/or for communication with an external computer, and/or for operation of the system.

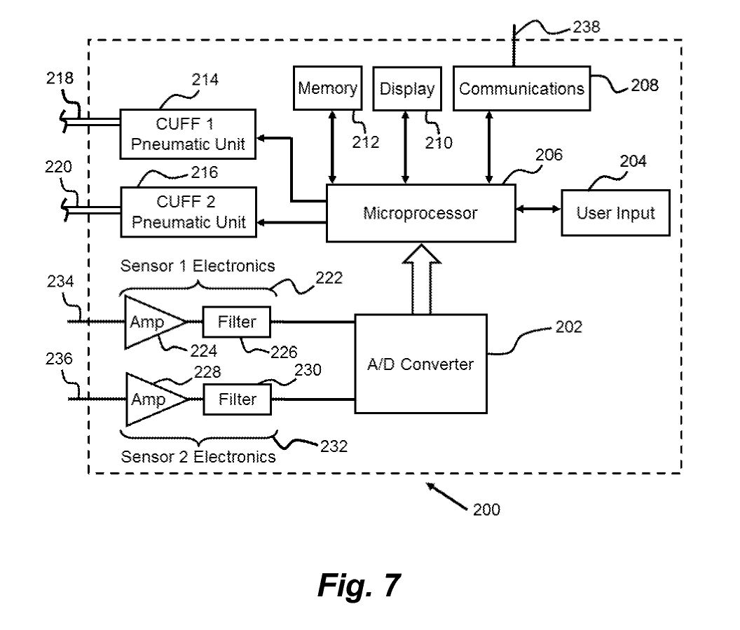

FIG. 7 provides a block diagram of a control 200 in accordance with one illustrative embodiment of the present invention. A microprocessor 206 optionally serves a central control and integration function controlling the various units/components therein. As illustrated in FIG. 7, the control unit includes, or is coupled to a pneumatic or hydraulic unit 214 (e.g., a unit comprising a pressure source 103 and/or a pressure controller 105) that operates to establish a substantially constant pressure in a cuff (cuff 1) via a hydraulic or pneumatic line 218. In certain embodiments, particularly where a pressure pulse transit time is to be determined, the control unit optionally includes, or is optionally coupled to a second pneumatic or hydraulic unit 216 (e.g., a unit comprising a pressure source 103 and/or a pressure controller 105) that operates to establish a substantially constant pressure in a second cuff (cuff 2) via a hydraulic or pneumatic line 218. It will be appreciated that the pneumatic or hydraulic control units can be used generally to inflate and/or deflate the cuffs as well.

Sensor electronics 222 are provided to send commands to sensor transducer and/or to read a signal from the pressure transducer monitoring pressure in the first cuff (cuff 1). Thus, in certain embodiments, a signal from a first pressure transducer in cuff 1 is transmitted along line 234 to sensor electronics 222, comprising for example, an amplifier 224, and/or a filter or signal conditioner 226 and/or any other electronics useful to drive, read, or transform the pressure transducer signal. An analogue to digital converter (A/D) 202 optionally converts the readings of the pressure transducer from cuff 1 and/or sensor electronics 222 into digital samples provided to microprocessor 206.

Where a second cuff is to be monitored, the control unit optionally further comprises sensor electronics 230 to send commands to sensor transducer and/or to read a signal from the pressure transducer monitoring pressure in a second cuff (cuff 2). Thus, in certain embodiments, a signal from a second pressure transducer in cuff 2 is transmitted along line 236 to sensor 1 electronics 232, comprising for example, an amplifier 228, and/or a filter or signal conditioner 230 and/or any other electronics useful to drive, read, or transform the pressure transducer signal. An analogue to digital converter (A/D) 202 optionally converts the readings of the pressure transducer from cuff 2 and/or sensor electronics 2232 into digital samples provided to microprocessor 206.

In illustrative embodiments, the pressure transducers comprise a sensor such as the Millar catheter pressure sensor (Mikro-tip, Millar Instruments, Houston, Tex.) or MEMS pressure sensor such as the NPC-1210 (GE Novasensor, Fremont, Calif.), but most low cost sensors used in automatic sphygmomanometers constitute suitable transducers.

Microprocessor 206 optionally also communicates with display 210, user input interface 204, and dynamic memory or static memory storage media 212 (e.g., disk drive, flash memory, optical memory, etc.). In some embodiments one or more communications lines 208 are used to communicate with an external computer or any other external unit. Power can be provided to the unit by an internal or external power supply that receives external power through a cable and/or through batteries.

In certain embodiments, the control unit 111/200 can be connected to a computer via Bluetooth, via a cable, and the like for configuration, control, and/or data download. In certain embodiments, the computer is integrated into the control unit and microprocessor 206 can function as the central processing unit of the computer, or another microprocessor is optionally present for such function. The computer can, for example, be dedicated for use with system 200, a personal computer in a physician's clinic, part of a hospital network and/or a remote computer connected, for example, through the internet, an intranet, or via a cell phone link. In certain embodiments, for example, a computer network connection can be used for may be used for receiving patient data and/or providing test results to remote locations. In some embodiments the computer manages a database of test results classified according to demographic and/or epidemiologic data for the purpose of determining endothelial dysfunction trends and/or for comparing current test results to previously acquired results from same or different patients. In some embodiments, the computer connects with a patient medical record system such as is maintained by a hospital, physician's office, HMO, PPO, and the like.

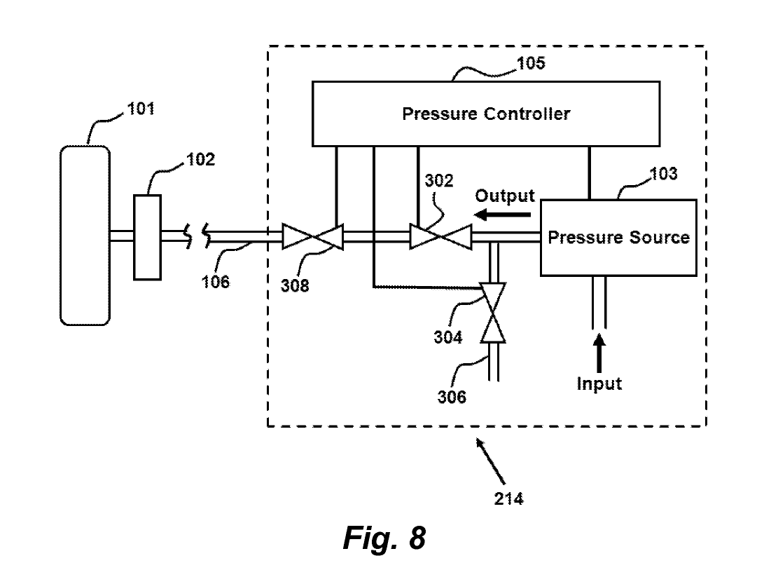

FIG. 8 provides a schematic view of one embodiment of a pneumatic/hydraulic unit 214 shown in FIG. 7. Pneumatic unit 214 includes a pressure source 103 configured to provide output pressure up to a pressure that completely occludes blood flow through a limb or portion of a limb (or other region of a body). Typically pressures can be delivered that range up to about 200 mmHg, up to about 250 mmHg, up to about 300 mmHg, up to about 350 mm Hg, up to about 380 mmHg, or up to about 400 mmHg or greater. Valve 302 optionally controls flow of a pressurized gas (e.g., air or other pressurized gas or gas mixture), or a pressurized fluid or gel from pressure source 103 to cuff 100. A valve 302 is optionally shut off after a desired substantially constant pressure is applied to the cuff. Another valve 304 is optionally provided to vent the cuff through outlet port/wasteline 306 to reduce pressure or deflate the cuff.

An optional valve 308 can be provided to restrict flow to the cuff and thereby slow the response time of the pneumatic/hydraulic unit so that pressure regulation does not substantially attenuate pulses produced in the cuff by cardiac activity. A pressure line 106 carries the gas, fluid, or gel to the cuff whereby the cuff is inflated or deflated. In certain embodiments the pressure line 106 is a narrow line that constricts flow thereby reducing the response time of the pneumatic/hydraulic unit. A pressure controller 105 is optionally incorporated into the pneumatic/hydraulic unit to regulate flow into and out of the pressure source and/or to regulate valves 306 and/or 304, and/or 302.

Any of the foregoing systems and devices can further include units to induce an artificial arterial pressure pulse. Such units include, but are not limited to a linear actuator, as described above (see, e.g., Maltz and Budinger supra.), a disk-type and a nozzle-flapper type electro-pneumatic converter (EPC) for the cuff-pressure control (see, e.g., Tanaka et al. supra), a standard cuff, and the like.

FIG. 9 provides flow chart illustrating typical acts performed in a measurement of the effect of a stimulus on endothelial function. The subject is typically allowed to rest (e.g., for at least 1 minute, at least 2 minutes, at least 3 minutes, at least 4 minutes, at least 5 minutes, at least 10 minutes, at least 15 minutes, etc.) to avoid the effect of transient activity of other stimulation on the measurement. The subject may be required to avoid eating, taking medicine, smoking and/or drinking coffee for certain periods of time (e.g., two hours or more before the test). The cuff or cuffs (e.g., depending on whether a transit time calculation is to be made) are affixed to the desired regions(s) of the subject (e.g., arm, leg, wrist, ankle, etc.). The blood pressure of the subject is optionally determined using any method known in the art and/or using the system itself. The cuff(s) are then inflated to a substantially continuous pressure at or below the measured diastolic pressure of the subject. Thus, in certain embodiment the cuffs are inflated to a pressure below the measured (or mean or median measured) diastolic pressure (e.g., not more than about 10 mm Hg below the diastolic pressure, or not more than about 15 mm Hg below the diastolic pressure, or not more than about 20 mm Hg below the diastolic pressure, or not more than about 25 mm Hg below the diastolic pressure, or not more than about 30 mm Hg below the diastolic pressure). A pressure pulse or series of pressure pulses resulting from one or more cardiac cycles is then recorded providing baseline pressure versus time data. The data is optionally processed to provide one or more parameters (e.g., maximum expansion, integrated pressure/time, maximum slope of pressure pulse, transit time of pulse from one cuff to a second cuff, etc.).

A stimulus is then applied to the subject. Any of a number of stimuli expected to alter endothelial function are contemplated. Such stimuli include, for example, occlusion of blood flow, and/or application of one or more drugs to the subject. Illustrative drugs include, for example, drugs that act as NO agonists (e.g. acetylcholine), β₂-adrenergic agonists such as albuterol, acoustic/mechanical tissue vibration, transcutaneous low frequency ultrasound (see, e.g., Iida et al. (2006) J. Amer. Coll. Cardiol., 48(3): 532-537), and the like. The contribution of basal NO release to basal vascular tone may be elicited by administering NO-synthase inhibitors such as L-NMMA and L-NAME. These agents may be administered via intra-arterial infusion (as is conventional practice) or by means of novel administration methods we have demonstrated involving nasal inhalation and ingestion. Endothelium-independent smooth muscle function may be evaluated by administration of NO-releasing drugs such as nitroglycerin and sodium nitroprusside.

In certain embodiments, the stimulus excludes occlusion and/or application of drugs. In certain embodiments the stimulus excludes occlusion and/or application of drugs that are NO agonists.

In certain embodiments the stimulus comprises acoustic/mechanical tissue vibration, or transcutaneous low frequency ultrasound.

A pressure pulse or a series of pressure pulses resulting from one or more cardiac cycles is then recorded providing stimulus-effected pressure versus time data. The data is again optionally processed to provide one or more parameters (e.g., maximum expansion, integrated pressure/time, maximum slope of pressure pulse, transit time of pulse from one cuff to a second cuff, etc.).

The baseline data or derived parameters is then compared to the stimulus-effected data or derived parameters to determine the presence, absence, and/or magnitude of the effect of the stimulus. In certain embodiments the results may be recorded in a database (e.g., in a medical record).

In certain embodiments the blood pressure can be eliminated and the cuffs simply inflated to a predetermined or arbitrary substantially constant pressure.

In certain embodiments when using occlusion as a stimulus, Alternatively to occluding the same artery on which the measurements are performed, a different artery connected to the measured artery, is occluded. For example, when the measurements are performed on the brachial artery, the occlusion may be applied to the radial and/or ulnar arteries. Ideally, when such a cuff is used to assess endothelial function, the occluding cuff is placed downstream of the points of measurement. This increases the contribution of NO-dependent mechanisms to the vasodilation that occurs, and minimizes the effects of tissue ischemia (which, are substantially mediated by other biochemical pathways not dependent on NO). The two cuffs may be integrated into a single entity containing two fillable air cavities. The upstream cavity is inflated only during the measurement intervals (to subdiastolic pressures), while the downstream cavity is used only for inducing endothelial stimulus via reactive hyperemia (inflated to suprasystolic pressures). In this way, the measurement is always obtained in an arterial segment that was not subject to ischemia.

The baseline phase measurement(s) optionally includes a plurality of rounds (e.g., 2-5 rounds), in each of which the pressure versus time data are recorded. The results of the plurality of measurement rounds can be optionally averaged to, in principle, reduce noise in the measurements. In addition to, or as an alternative, other noise reducing statistical methods can be utilized. Alternatively, in certain embodiments a single measurement is performed in order to limit the time required for the measurement session. Several of the earliest baseline measurement rounds may be discarded according to a predetermined protocol in order to minimize any initial deformation of the limb cross section that may occur during the first measurements.

In certain embodiments the stimulus-effected measurements are made a predetermined time after application of the stimulus, e.g., when the stimulus effect is expected to be maximal.

In various embodiments repeated measurement rounds can be made after periods of reduced or eliminated cuff pressure to prevent the repeated measurement rounds from inducing hyperemia which would influence the measurements and/or prevents the repeated measurement rounds from causing discomfort to the patient.

As indicated above, in certain embodiments a score or derived parameter representative of endothelial function is determined based on the effect (or absence of effect) of the stimulus (depending on the stimulus used). In certain embodiments the score is compared to a threshold and accordingly a binary diagnosis is provided (e.g., normal, abnormal). In some embodiments, the threshold depends on one or more attributes of the patient, such as gender, height, weight and/or age. Alternatively or additionally, a multi-level diagnosis is provided, for example giving a value in percentages or other units. The multi-level diagnosis is optionally determined by comparing the score to an array of thresholds or to a “standard” curve.

As mentioned above, during the test session, between the base line phase and the stimulus effected measurement, the subject preferably remains at rest, so as to minimize the difference in conditions between the measurements. Alternatively or additionally, the results are corrected for changes in the conditions between the phases.

As indicated above, in some embodiments, the difference in the baseline and stimulus effected parameters is calculated by determining an envelope of the measurements and finding a maximum value with the envelope to use as the basis of the parameter calculation. In certain embodiments the maximal difference in the value of the parameter(s) between the baseline and stimulus-effected parameters is determined. The calculation is performed using any method known in the art, such as using a fitting method which finds a maximal difference over a single cardiac cycle, or over a plurality of cardiac cycles (e.g., 2, 3, 4, 5, 6, 7, 8, 9, 10, 11, 12, 13, 14, 15, 20, 25, 30, or more cardiac cycles).

As mentioned above, the systems described herein can be used to determine the blood pressure of the subject patient, during endothelial function tests or separately.

Typically, such measurements can be made by inflating the cuff to a pressure above the systolic pressure of the subject and the air pressure of the cuff is deflated to below the diastolic blood pressure of the patient. During the air pressure deflation, pressure transducer 102 registers the changes in the pressure of measurement cuff 100. The resulting data is then analyzed to find the systole (SYS), and/or diastole (DIA) pressures, using any of the methods known in the art for oscillatory blood pressure measurement.

It is noted that a poor dilatation functioning may occur due to arteriosclerosis of a specific artery. In order to prevent identification of endothelial dysfunction in subjects that have local arteriosclerosis in a single artery but do not suffer from endothelial dysfunction, in some embodiments the methods described herein are repeated on another artery of the subject, for example on the opposite arm. If dysfunction identified for one artery but not the other, the subject is identified as not having endothelial dysfunction and/or is sent for additional tests.

In some embodiments, the microprocessor 206 and/or separate computer is programmed to carry out a complete test session automatically without requiring instructions from a human operator. Optionally, control unit 200 checks that the conditions are proper and stops the test session if a problem is detected, for example when signal is detected, when pressure exceeds a threshold, or when no sensible data is produced perhaps due to erratic or significant oscillations in the blood pressure of the subject during the test.

Alternatively, the operation sequence of a test session may be partially or entirely human operated. For example, each measurement phase may be controlled automatically by microprocessor 206, while the initiation of each phase is controlled by a human operator. Optionally, an operator may program operation sequences through a computer or other device. Alternatively or additionally, required operation sequences are preprogrammed into microprocessor 206 at the time of manufacture.

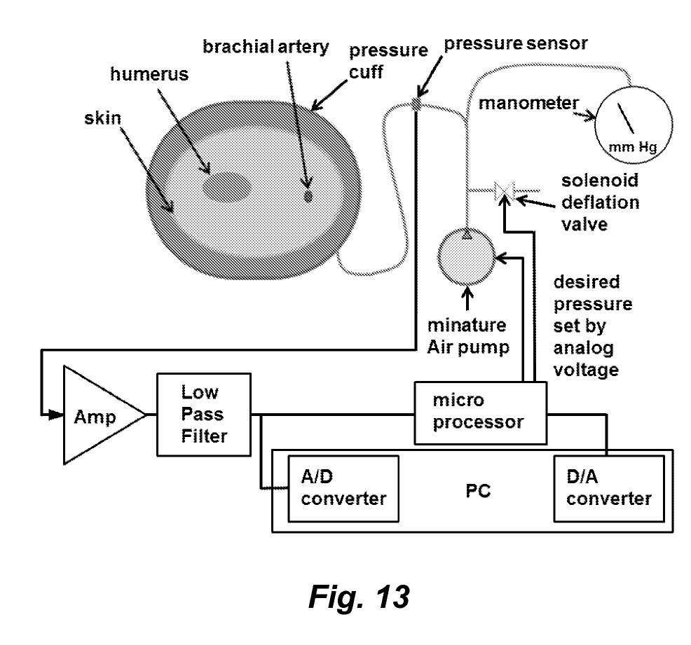

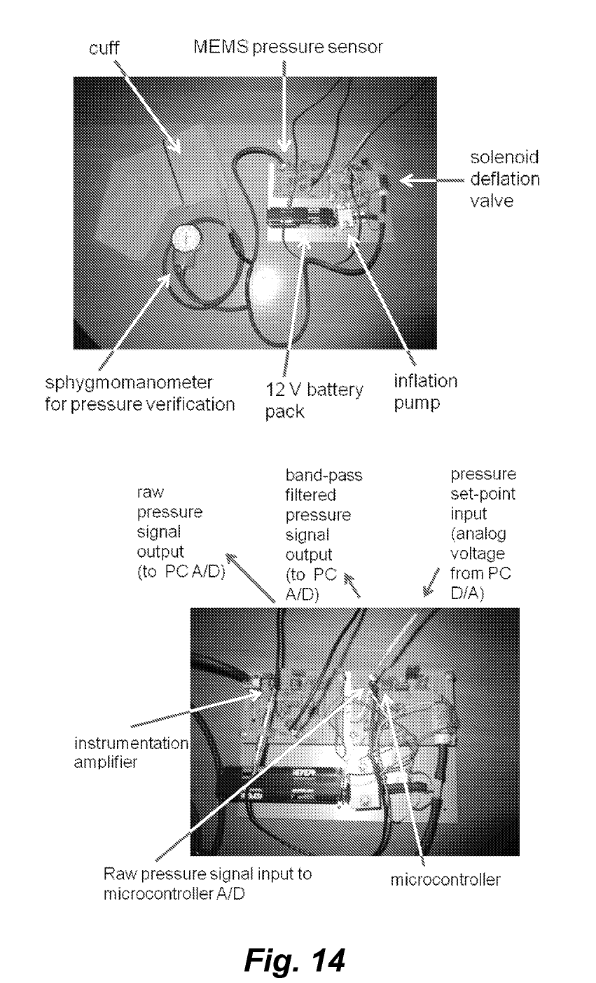

In certain embodiments the prototype illustrated in FIG. 1 may use an expensive and bulky pneumatic regulator to produce constant pressure in the cuff during measurement. In contrast, one illustrative and less expensive portable prototype, is shown in the schematic diagram of FIG. 13, and photographs of FIG. 14. This version can use a miniature pump and solenoid valve to control cuff pressure. Since the pump and solenoid valve provide on-off control, the pressure in the cuff generally falls with time as the tissue under the cuff displaces. While this is a disadvantage, there are compelling reasons to use on-off control: 1) There is no need for an expensive pressure regulator and compressed air source; 2) The pump preferably does not operate during measurement as it introduces noise into the signal; 3) The component count is smaller and the cost is much lower; and 4) Standard pumps and valves employed in home blood pressure measurement systems can be used. Since the pump and valve may be actuated during a measurement interval, the recorded signal may be contaminated with noise. For offline processing applications, this can be removed using low-pass filtering of the recorded time-series. For online processing, the times of actuation can be fed into the data analysis algorithm to ensure this noise does not confound the analysis.

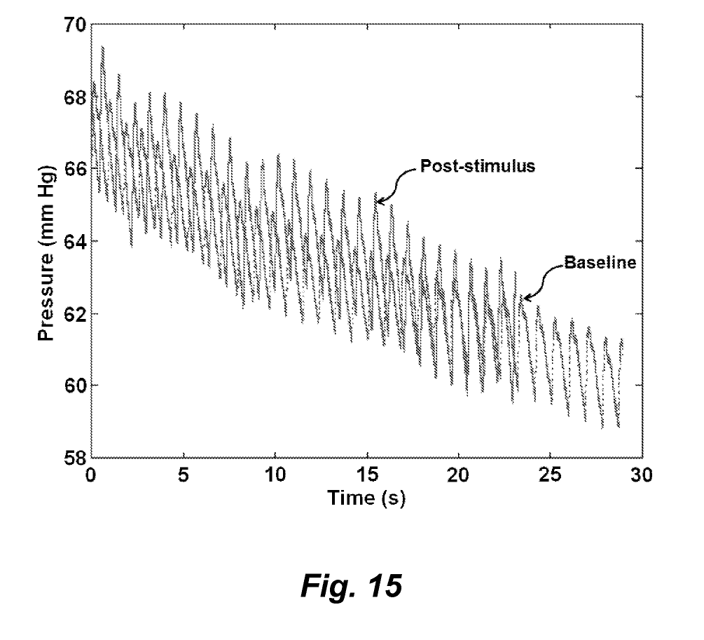

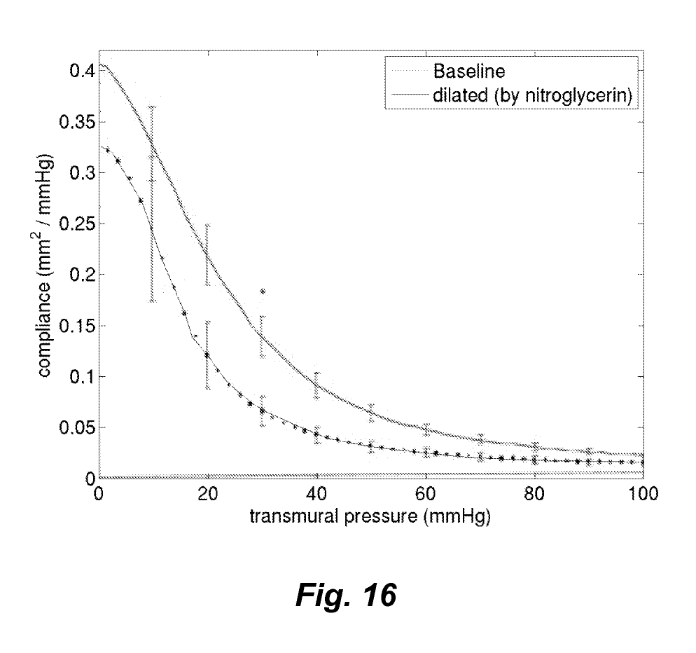

To address this issue, we have developed a method of data analysis that improves the accuracy of a system with on-off control to the extent that impressive results such as those shown in Table 2 are possible. Consider FIG. 15, which demonstrates the typical fall in pressure during a measurement interval. The curves in FIG. 16 illustrate the effect of article unloading on arterial compliance (Bank et al. (1999) Circulation, 100: 41-47). A decrease in unloading pressure of 8 mmHg (as seen in FIG. 15) can impact compliance significantly when the transmural pressure is small (10-20 mmHg in our case at diastole). Clearly, when comparing pulse properties such as amplitude and maximum upward slope, before and after stimulus, it is preferably to compare pulses measured at like cuff pressures. For example, in FIG. 15, while it is appropriate to directly compare the post-stimulus and baseline at t=20 s, this is not the case at t=10 s. In the former case, the cuff measurement pressure is similar, but in the latter case, it is larger during the post-stimulus series than during the baseline series.

One illustrative approach to this issue is to “histogram” the pulses by pressure, using a binning statistic such as the mean, median, minimum or maximum pressure during the pulse. Pulses in each histogram bin from the baseline and response series are compared and the fractional change is computed for each bin. A weighted average of the bins is taken, where the weights are proportional to the number of pulses in each bin and the confidence in each measurement.

In cases where the ranges of pressure do not completely overlap, curves such as those shown in FIG. 16 can be used to adjust the data so all pulses can be compared.

From our human subject studies, it is apparent that oscillatory subject motion such leg shaking can introduce spurious waveforms that may be interpreted as pulses. This can be addressed by means of software and/or hardware. One software approach is to perform real-time analysis of the incoming pressure signal and detect anomalies. In a hardware approach, an accelerometer can be placed on the cuff, on the cuff tube or in the instrument itself to detect vibrations that cannot be easily filtered out (e.g., those that are in the same frequency band as the signal of interest). The system can then generate an alert to the user indicate that vibration is present and may abort the measurement if vibration does not cease.

It will be appreciated that the above described methods and apparatus may be varied in many ways, including, changing the order of acts of the methods, and the exact implementation used for the apparatus. It should also be appreciated that the above described methods and apparatus are to be interpreted as including apparatus for carrying out the methods and methods of using the apparatus.

The devices and methods have been described herein using non-limiting detailed descriptions of embodiments thereof that are provided by way of example and are not intended to limit the scope of the invention. For example, rather than performing the endothelial dysfunction test on the arm, the method may be performed on a subject's leg.

In addition, while the methods are described with reference to humans, the term mammal is intended to include humans as well as non-human mammals (e.g., non-human primates, canines, equines, felines, porcines, bovines, ungulates, largomorphs, and the like).

It should be understood that the methods and apparatus described herein measure endothelial dysfunction by means of measuring the consequences of vascular smooth muscle relaxation, and that these methods may therefore be applied to measure smooth muscle function simply by substituting the endogeneous source of nitric oxide (endothelial NO release) with an exogenous source, such as sublingual nitroglycerin.

It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

### EXAMPLES

The following examples are offered to illustrate, but not to limit the claimed invention.

### Example 1

### Validation of Measurement of Endothelial Function

One way to determine the value of a new measure is to determine to what degree it is correlated to a “gold standard” measurement. In the case of endothelial function, the gold standard is dilation of coronary arteries in response to infused acetylcholine (ACh). This procedure is invasive, expensive and suitable only for diseased patients undergoing cardiac catheterization.

In the evaluation of a previous instrument that we developed for the assessment of endothelial function, we determined the correlation between our method and ultrasound-based FMD measurements in the brachial artery. While non-invasive, FMD studies are technically difficult and produce measurements with high variance.

We contend that such studies are unnecessary in order to make a determination that a method is effective in assessing endothelial function. Physical methods for arterial EF evaluation typically measure changes in the material properties of the artery. The changes that occur in response to endogenous release of NO are similar in nature and magnitude to those that occur following administration of exogenous NO via agents such as nitroglycerin (NG). As a consequence, if we can show that a measurement method is sensitive to vasorelaxation induced by NG, we can assume that the technique will also be sensitive to endothelium-mediated vasorelaxation. A major advantage of this method is that response to NG is intact even in individuals with endothelial dysfunction, so there is no need to perform a correlation analysis between two measurements.

To further strengthen the case, the measurement method should demonstrate sensitivity to RH-induced vasorelaxation in individuals who would be expected to have intact endothelial response.

We examined three individuals in the age-range 28-38. Table 1 lists the subject characteristics. All subjects had Framingham risk scores of 1% or less, and had no history of cardiovascular disease. Each individual was assessed at least three times before and after RH induced by five minutes of suprasystolic cuff occlusion. At least one additional measurement was made using the same protocol, except without cuff inflation. Sensitivity to a 0.4 mg dose of sublingual NG was assessed three times in two individuals.

TABLE 1
Subject characteristics. (NS: no stimulus, RH: reactive hyperemia, NG:
nitroglycerin).

| | | | | Number of |
|---|---|---|---|---|
| | | | Framingham | Studies |
| Subject | Gender | Age | Score | NS/RH/NG |
| Subject 1 | Male | 38 | 1% | 3/3/3 |
| Subject 2 | Female | 38 | <1% | 4/3/3 |
| Subject 3 | Male | 28 | <1% | 1/3/0 |

Each individual was assessed at least three times before and after RH induced by five minutes of suprasystolic cuff occlusion. At least one additional measurement was made using the same protocol, except without cuff inflation.

Sensitivity to a 0.4 mg dose of sublingual NG was assessed three times in two individuals. An additional study was performed in each of these same two individuals using the same protocol, with no NG administration.

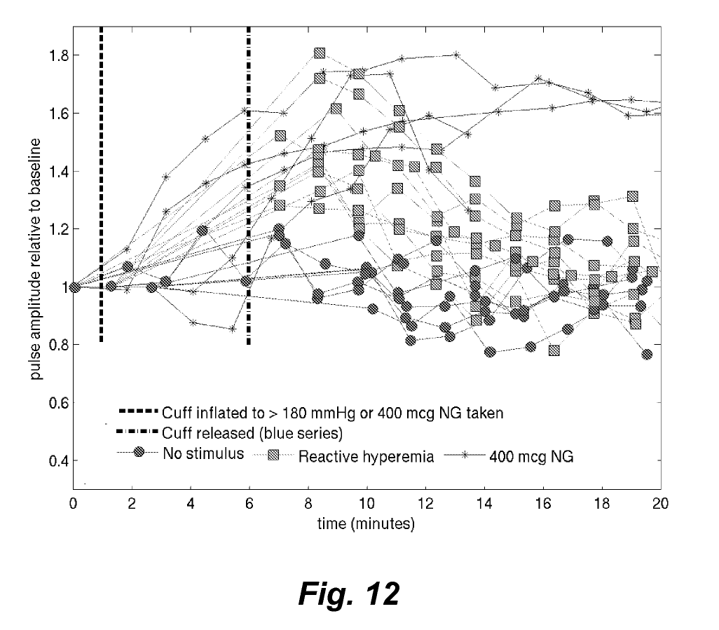

We studied a single quantity relating to the recorded pressure data: the pulse amplitude, which we posit is proportional to the arterial area. During the post stimulus interval, pressure data from the cuff were recorded approximately every 80 seconds, for a period of 30 seconds. During each recording interval, the cuff was inflated to 70 mmHg, which was always below the measured diastolic pressure of the subject. To quantify the observed response, we divided the mean of the pulse quantity (in this case, amplitude) during the response interval by the mean value of the same quantity during the baseline interval. FIG. 12 illustrates the results.

### Results

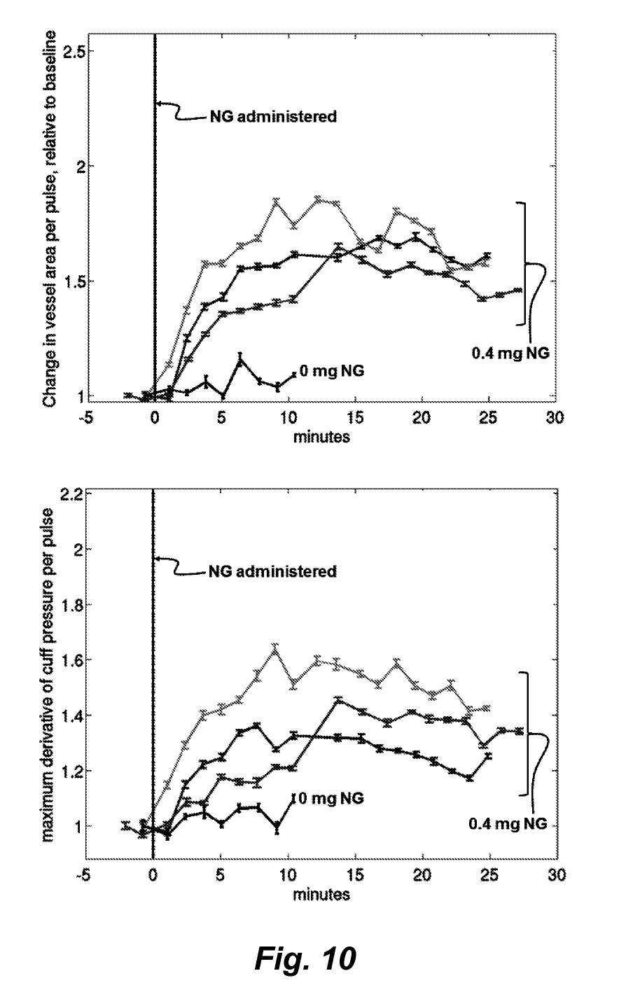

FIG. 10 illustrates the responses obtained in response to NG. In all cases where NG was administered, increases of more than 30% are observed in both metrics. Only relatively small baseline variation is evident when no NG was given. In the time interval from 6 minutes to 20 minutes, there is a large and persistent difference between the NG responses and the NS responses

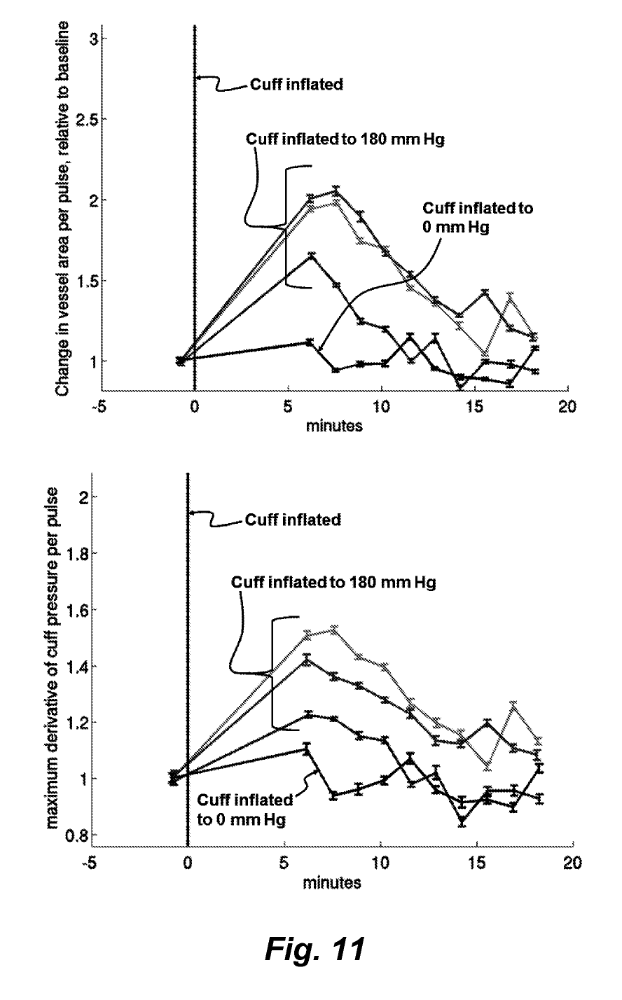

FIG. 11 shows responses to RH induced by a five-minute cuff occlusion in a 28 year-old male. In all three cases, responses to RH significantly exceed the case where no RH was induced. The area-based measurement appears more sensitive and consistent than the gradient based (compliance measurement). By both measures, endothelial function appears impaired in the first RH series relative to the other two RH series. For the RH studies, there is no overlap between the RH and NS data responses during the four minutes following cuff release.

In Table 2, we calculate the maximum response for each stimulus and evaluate the statistical significance of the change relative to the NS case (one-tail Student's t test). Values of p<0:05 were considered significant.

TABLE 2
Statistical analysis of amplitude response.

| | Mean ± SEM of maximum | p-value |
|---|---|---|
| Stimulus | Response over all data sets | versus NS |
| RH | 1.51 ± 0.052 | 1.19 × 10⁻⁵† |
| NG | 1.70 ± 0.036 | 6.25 × 10⁻⁶† |
| NS | 1.01 ± 0.068 | N/A |

### CONCLUSION

While the current sample size of three subjects is small, the use of repeated measurements per subject has allowed us to demonstrate with great statistical certainty that the proposed measurement device is capable of detecting changes due to RH (p=1.19×10⁻⁵) and NG (p=6.25×10⁻⁶) in all subjects on all occasions. This statistical analysis invalidates the null hypothesis that RH or NG evoke equal responses to NS in this set of subjects. The fact that there is no overlap between NS and either of the response classes in FIG. 12 is a truly impressive result.

As discussed above, since NG response is intact in almost all individuals, little is gained in examining a larger population. Our results show that the sensitivity of the method is approximately 5 times greater than that of ultrasound-based imaging of arterial diameter in response to flow-mediated dilation (FMD due to RH). This is based on a comparison of the 51% mean maximum increase in pulse amplitude over baseline versus the approximate 10% brachial artery diameter change representative of an intact endothelial response in B-mode ultrasound FMD studies in the literature.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of assessing endothelial function in a mammal, said method comprising:
applying to the artery a substantially constant external pressure that causes the tension in the artery wall to decrease, where said pressure is provided via a cuff adjacent to and/or around a region of the mammal's body;
determining, over the course of one or more cardiac cycles, changes in pressure in said cuff resulting from cardiac activity of said mammal, or an artificially induced arterial pulse to establish a baseline value for a parameter related to endothelial function in said mammal;
applying a stimulus to said mammal; and
determining, over the course of one or more cardiac cycles, changes in pressure in said cuff resulting from cardiac activity of said mammal, or an artificially induced arterial pulse, to determine a stimulus-effected value for a parameter related to endothelial function in said mammal;
wherein said baseline value is determined from measurements made when said mammal is not substantially effected by said stimulus; and
wherein differences in said baseline value and said stimulus-effected value provide a measure of endothelial function in said mammal.

2. The method of claim 1, wherein said establishing a baseline value comprises establishing a baseline value for an artificially induced arterial pulse.

3. The method of claim 1, wherein said determining a stimulus-effected value comprises determining a stimulus-effected value for an artificially induced arterial pulse.

4. The method of claim 1, wherein said establishing a baseline value comprises establishing a baseline value for changes in pressure resulting from cardiac activity of said mammal.

5. The method of claim 1, wherein said determining a stimulus-effected value comprises establishing a stimulus-effected value for changes in pressure resulting from cardiac activity of said mammal.

6. The method of claim 1, wherein said substantially constant pressure is applied by a pressurized cuff disposed around an arm or leg of said mammal.

7-8. (canceled)

9. The method of claim 1, wherein said substantially constant external pressure is maintained by a system that monitors and adjusts the pressure in said cuff and whose response time is sufficiently slow so that the changes in pressure resulting from said cardiac activity are not substantially attenuated by said system.

10. (canceled)

11. The method of claim 1, wherein said substantially constant external pressure is maintained by setting the pressure in said cuff to a value and not altering external pressure applied to said cuff during the measurements of pressure variations due to said cardiac activity.

12. The method of claim 1, wherein applying the pressure to the artery comprises applying a local pressure that does not substantially affect other blood vessels in a same limb as the artery.

13. The method of claim 1, wherein applying the external pressure to the artery comprises applying a pressure that affects an entire cross-section of a limb including the artery.

14. The method of claim 1, wherein said substantially constant external pressure is equivalent to or below the average diastolic pressure measured for said subject.

15-21. (canceled)

22. The method of claim 1, wherein said determining comprises:
integrating the value of a pressure change over time (calculating the area under a pressure/time curve) for one or for a plurality of cardiac cycles to determine an integrated pressure value; and/or
determining the maximum, or a certain percentile rank of the derivative of the pressure versus time wave form on the rising edge of a pressure pulse for one or for a plurality of cardiac cycles to determine a compliance value.

23-27. (canceled)

28. The method of claim 1, wherein applying the stimulus comprises restricting flow of blood to the limb by occlusion of a blood vessel.

29-30. (canceled)

31. The method of claim 28, wherein the same cuff is used to occlude the blood vessel and to apply the pressure on the artery.

32-41. (canceled)

42. The method of claim 1, wherein said stimulus comprises low intensity ultrasound.

43. (canceled)

44. A method of assessing endothelial function in a mammal, said method comprising:
applying to the artery at a first location a substantially constant external pressure that causes the artery to fully or partially collapse, where said pressure is provided via a first cuff adjacent to and/or around a region of the mammal's body;
applying to the artery at a second location a substantially constant external pressure that causes the artery to fully or partially collapse, where said pressure is provided via a second cuff adjacent to and/or around a region of the mammal's body;
determining, over the course of one or more cardiac cycles, changes in pressure in said first cuff resulting from cardiac activity of said mammal or an artificially induced arterial pulse, determining, over the course of one or more cardiac cycles, changes in pressure in said second cuff resulting from cardiac activity of said mammal or an artificially induced arterial pulse; and calculating a baseline transit time of a pressure pulse from said first cuff to said second cuff to establish a baseline value for a parameter related to endothelial function in said mammal;
applying a stimulus to said mammal;
determining, over the course of one or more cardiac cycles, changes in pressure in said first cuff resulting from cardiac activity of said mammal or an artificially induced arterial pulse, determining, over the course of one or more cardiac cycles, changes in pressure in said second cuff resulting from cardiac activity of said mammal or an artificially induced arterial pulse; and calculating the stimulus-effected transit time of a pressure pulse from said first cuff to said second cuff to establish a stimulus-effected value for a parameter related to endothelial function in said mammal;
wherein said baseline value is determined from measurements made when said mammal is not substantially effected by said stimulus; and
wherein differences in said baseline value and said stimulus-effected value for the transit time provide a measure of endothelial function in said mammal.

45. The method of claim 44, wherein said baseline transit time is calculated for an artificially induced arterial pulse.

46. The method of claim 44, wherein said stimulus-effected transit time is calculated for an artificially induced arterial pulse.

47-84. (canceled)

85. An apparatus for assessment endothelial function in a mammal comprising:
a measurement cuff adapted to apply a substantially constant pressure to an artery in said mammal;
a measurement unit adapted to detect and quantify over one or more cardiac cycles, pressure pulses in said cuff while said substantially constant pressure is applied;
a controller that is adapted to apply to the cuff a said substantially constant pressure where said controller monitors and adjusts the pressure in said cuff and whose response time is sufficient slow so that the changes in pressure resulting from said cardiac cycles are not substantially attenuated by said system, and/or that is adapted to control a pressure source and a valve to provide on-off control of the pressure in said cuff; and
a processor adapted to analyze, and/or store, and/or compare values determined from said pressure pulses in at least two measurement rounds.

86. The apparatus of claim 85, wherein said controller is configured to monitor and adjust said substantially constant pressure at a response time sufficiently slow so that said pressure changes resulting from said cardiac activity are attenuated by less than 10%.

87. The apparatus of claim 85, wherein said controller is configured to maintain said substantially constant external pressure by setting the pressure in said cuff to a value and not altering external pressure applied to said cuff during the measurements of pressure variations due to said cardiac activity.

88-108. (canceled)

109. An apparatus for assessment endothelial function in a mamma comprising:
a first measurement cuff adapted to apply a substantially constant first pressure to an artery in said mammal;
a second measurement cuff adapted to apply a substantially constant second pressure to an artery in said mammal;
a measurement unit adapted to detect and quantify over one or more cardiac cycles, pressure pulses in said first cuff and/or said second cuff while said substantially constant pressure is applied;
a controller adapted to apply to said first cuff said substantially constant first pressure and/or said second cuff said substantially constant second pressure where said controller monitors and adjusts the pressure in said first cuff and/or said second cuff and whose response time is sufficient slow so that the changes in pressure in said first cuff and said second cuff resulting from said cardiac cycles are not substantially attenuated by said system, and/or where said controller is configured to provide simple on-off pressure control; and
a processor configured to monitor pressure pulses in said first cuff and in said second cuff, identify corresponding pulses in said first and said second cuff and calculate a transit time for the pressure pulse in an artery from the location of said first cuff to said second cuff.

110-134. (canceled)
